(19) United States
(12) Patent Application Publication
(10) Pub. No.: US 2026/0315740 A1
(43) Pub. Date: Oct. 15, 2026

(54) CATHETERIZATION WITH AUTOMATIC INSERTION AND ROTATION OF NEEDLE AND RELEASE OF FUNCTIONAL ELEMENT

(71) Applicants: BRACCO IMAGING S.P.A., Milano (IT); FONDAZIONE ISTITUTO ITALIANO DI TECNOLOGIA, Genova (IT)

(72) Inventors: Alperen ACEMOGLU, Genova (IT); Maria KOSKINOPOULOU, Genova (IT); Leonardo Serra DE MATTOS, Genova (IT)

(21) Appl. No.: 19/117,208

(22) PCT Filed: Oct. 30, 2023
(86) PCT No.: PCT/EP2023/080191

§ 371 Date: Mar. 31, 2025

(30) Foreign Application Priority Data
Oct. 28, 2022 (IT) 102022000022212

(51) Int. Cl. A61M 25/01; A61B 17/34; A61B 34/20; A61B 34/30; A61M 25/06

(52) U.S. Cl. CPC A61M 25/0113; A61M 25/0606; A61B 2017/3407; A61B 2034/2057; A61B 2034/303; A61M 2025/0166

(57) ABSTRACT

A solution is proposed for accessing a target region (103) within a body-part (106) of a patient. A corresponding catheterization device (400; 500a; 500b) has a support element (142) for a functional element (115; 118) being a needle (118) or a catheter (115) mounted thereon. A curved guide (405) arranged on a slide (141) is used to guide the support element (142). An actuator (148) translates the slide (141) for causing the needle (118) to penetrate the body-part (106). A further actuator (410) rotates the support element (142) for reducing an angle between the needle (118) and the body-part (106) in response to a reaching of the target region (103). A still further actuator (154; 254) releases the functional element (115; 118) from the support element (142) in response to a completion of its rotation. A robotic system (600) comprising the catheterization device (400; 500a; 500b) is also proposed. Moreover, a corresponding method (900) for controlling the catheterization device (400; 500a; 500b) is proposed. A computer program and a corresponding computer program product for implementing the method (900) are further proposed. A method for inserting the catheter into the target region is also proposed.

### TECHNICAL FIELD

The present disclosure relates to the field of medical equipment. More specifically, this disclosure relates to catheterization devices.

### BACKGROUND

The background of the present disclosure is hereinafter introduced with the discussion of techniques relating to its context. However, even when this discussion refers to documents, acts, artifacts and the like, it does not suggest or represent that the discussed techniques are part of the prior art or are common general knowledge in the field relevant to the present disclosure.

Catheterization is a very common medical procedure that is used to provide direct access to a target region within a body part of a patient (such as in peripheral/deep catheterization procedures). Each catheterization procedure involves the insertion of a catheter into the target region (for example, a blood vessel). For this purpose, a needle is always used to penetrate the body-part until reaching the target region. Particularly, in a peripheral catheterization procedure the needle carries the catheter with it, whereas in a deep catheterization procedure the needle is used to provide a guide for a subsequent insertion of the catheter.

Catheterization procedures are normally performed manually by practitioners. However, the (manual) catheterization procedures have a very high failure rate, ranging from 45% to 60%. Therefore, in a substantial number of cases the corresponding practitioner needs two or more attempts to perform each catheterization procedure successfully. This is especially true in case of patients with medical situations leading to difficult access to the corresponding target regions. Typical examples are patients being very young (children), being very old, with dark complexion or with chronic conditions (such as obesity, diabetes, parenteral drug abuse, undergoing chemotherapy, and so on). For example, in these kind of patients the practitioner faces difficulties in identifying the blood vessel that is most suited for the catheterization procedure. Moreover, the needle may not be inserted in a correct position, which happens when the insertion is too shallow, too deep or is executed with a wrong angulation. Therefore, the needle may cause damages to the patient, for example, when it completely crosses the blood vessel from one side to its opposite side. In any case, a result of the catheterization procedure strongly depends on skills (and attention) of the practitioner. All of the above may lead to unwanted (more or less serious) consequences for the patients, such as clotting, allergic reactions, appearance of black spots, darkening of veins due to venous scarring and hemorrhage/gangrene due to arterial damages.

Therefore, catheterization devices have been proposed to help practitioners in performing the catheterization procedures.

For example, WO-A-2018219842 discloses a catheterization device of handheld type. The needle and the catheter are carried by a shaft, which is normally coupled with a casing, so that they are moved by a practitioner as a single piece. The catheterization device detects when a tip of the needle has reached a target position within the body-part, according to a signal that measures a physical characteristic of a material at the tip of the needle. In response thereto, the shaft (and then the needle and the catheter) are decoupled from the casing. Therefore, any possible further forward movement of the casing caused by the practitioner is no longer transmitted to the shaft, and therefore the tip of the needle remains at the target position. WO-A-2018219842 also discloses a specific embodiment wherein the catheterization device has a motor that may be used to insert the needle into the vein. The same motor may also be used to retract the needle while the catheter is inserted into the vein by the practitioner moving the casing once the catheterization device has detected that the tip of the needle has reached the target position within the body-part. Moreover, WO-A-2018219842 discloses another specific embodiment wherein the catheterization device has an actuator that may insert the needle and the catheter. Once the catheterization device has detected that the tip of the needle has reached the target position within the body-part, a mechanism is enabled that moves the needle backwards while the catheter moves forwards.

WO-A-93/05832 discloses another catheterization device of hand-held type. A practitioner may use one hand to grasp and position the catheterization device during the insertion of the needle. Once the needle has entered a blood vessel, the catheterization device automatically advances the catheter so as to release it (by means of a spring, compressed gas or a pair of magnets); this is triggered manually by the practitioner or automatically in response to the detection of the penetration of the blood vessel by a corresponding sensor.

EP-A-2306920 discloses an image guided robotic device with a first motor for advancing/retracing a needle and a second motor for advancing/retracing a catheter relative to the needle so as to move them in opposite directions thereby separating the catheter from the needle; insertion of the needle into a vessel is stopped when the needle has penetrated the vessel to a proper depth identified by a crosshair in corresponding acquired images, and the catheter is released after a guidewire has extended past a tip of the needle. An actuator for deploying the needle/catheter is connected to a console by using an arcuated support arm; the actuator pivots relative to the console to a proper angle for insertion of the needle/catheter, after that the first motor is activated to advance the needle/catheter.

EP-A-3831303 discloses an autonomous intravenous insertion system with a motor for advancing a needle and a catheter, and another motor that releases the catheter from the needle after its insertion.

US-A-2021/369297 discloses a vacuum-assisted insertion device with a needle drive assembly that moves a needle and a catheter; the needle drive assembly comprises a releasing mechanism for detaching the catheter from the needle after its insertion. Moreover, this document also generically mentions the possibility of pivoting the needle about a tip thereof (co-axially with a vessel) once the tip of the needle enters the lumen of the vessel.

However, further improvements would be desirable to the catheterization devices in order to facilitate a task of the practitioners.

### SUMMARY

A simplified summary of the present disclosure is herein presented in order to provide a basic understanding thereof; however, the sole purpose of this summary is to introduce some concepts of the disclosure in a simplified form as a prelude to its following more detailed description, and it is not to be interpreted as an identification of its key elements nor as a delineation of its scope.

In general terms, the present disclosure is based on the idea of providing automatic insertion and rotation of a needle and release of a functional element.

Particularly, an aspect provides a catheterization device for accessing a target region within a body-part of a patient. The catheterization device has a support element for a functional element being a needle or a catheter mounted thereon. A curved guide arranged on a slide is used to guide the support element. An actuator translates the slide for causing the needle to penetrate the body-part. A further actuator rotates the support element for reducing an angle between the needle and the body-part in response to a reaching of the target region. A still further actuator releases the functional element from the support element in response to a completion of its rotation.

A further aspect provides a robotic system comprising the catheterization device.

A further aspect provides a control method for controlling the catheterization device.

A further aspect provides a software program for implementing the control method.

A further aspect provides a corresponding software program product.

A further aspect provides a corresponding catheterization method.

More specifically, one or more aspects of the present disclosure are set out in the independent claims and advantageous features thereof are set out in the dependent claims, with the wording of all the claims that is herein incorporated verbatim by reference (with any advantageous feature provided with reference to any specific aspect that applies mutatis mutandis to every other aspect).

### BRIEF DESCRIPTION OF THE DRAWINGS

The solution of the present disclosure, as well as further features and the advantages thereof, will be best understood with reference to the following detailed description thereof, given purely by way of a non-restrictive indication, to be read in conjunction with the accompanying drawings (wherein, for the sake of simplicity, corresponding elements are denoted with equal or similar references and their explanation is not repeated, and the name of each entity is generally used to denote both its type and its attributes, like value, content and representation). In this respect, it is expressly intended that the drawings are not necessary drawn to scale (with some details that may be exaggerated and/or simplified) and that, unless otherwise indicated, they are merely used to illustrate the structures and procedures described herein conceptually. In addition, orientations and related position references (such as front, rear, upper, lower, lateral and so on) are to be understood in relation to a condition of use of the corresponding entities. Particularly:

FIG. 1A-FIG. 1D show a schematic representation of operation of a catheterization device,

FIG. 2A-FIG. 2D show a schematic representation of operation of another catheterization device,

FIG. 3 shows a pictorial representation of a catheterization device of hand-held type,

FIG. 4A-FIG. 4D show a schematic representation of the principles of the solution according to an embodiment of the present disclosure,

FIG. 5A-FIG. 5C and FIG. 5D-FIG. 5E show operation of different catheterization devices of hand-held type according to an embodiment of the present disclosure,

FIG. 6 shows a pictorial representation of a robotic system comprising the catheterization device according to an embodiment of the present disclosure,

FIG. 7 shows a pictorial representation of a catheterization device of end-effector type,

FIG. 8A-FIG. 8D show operation of a catheterization device of end-effector type, and

FIG. 9A-FIG. 9B show an activity diagram describing the flow of activities relating to an implementation of the solution according to an embodiment of the present disclosure.

### DETAILED DESCRIPTION

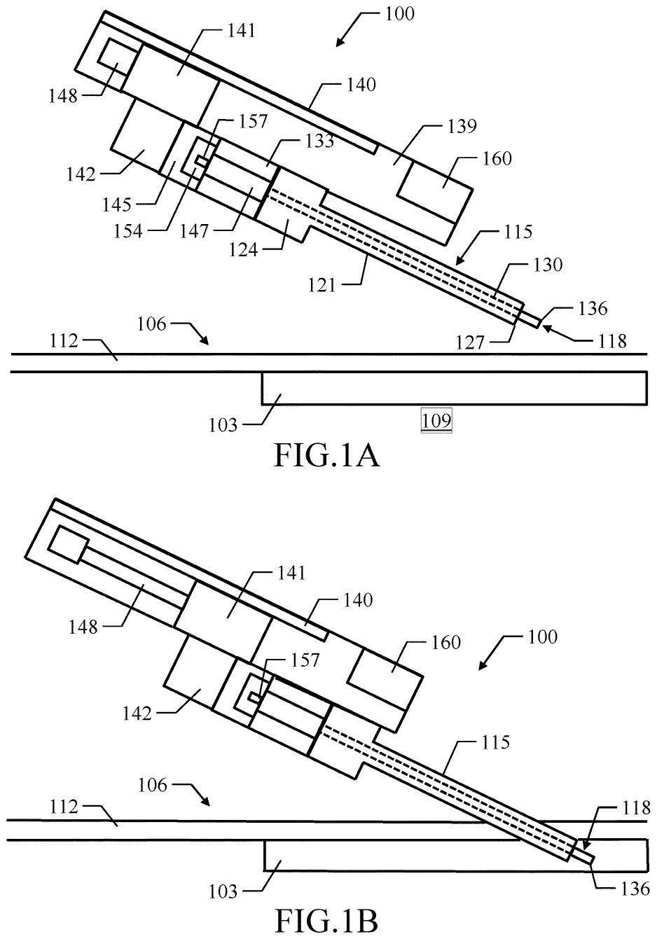

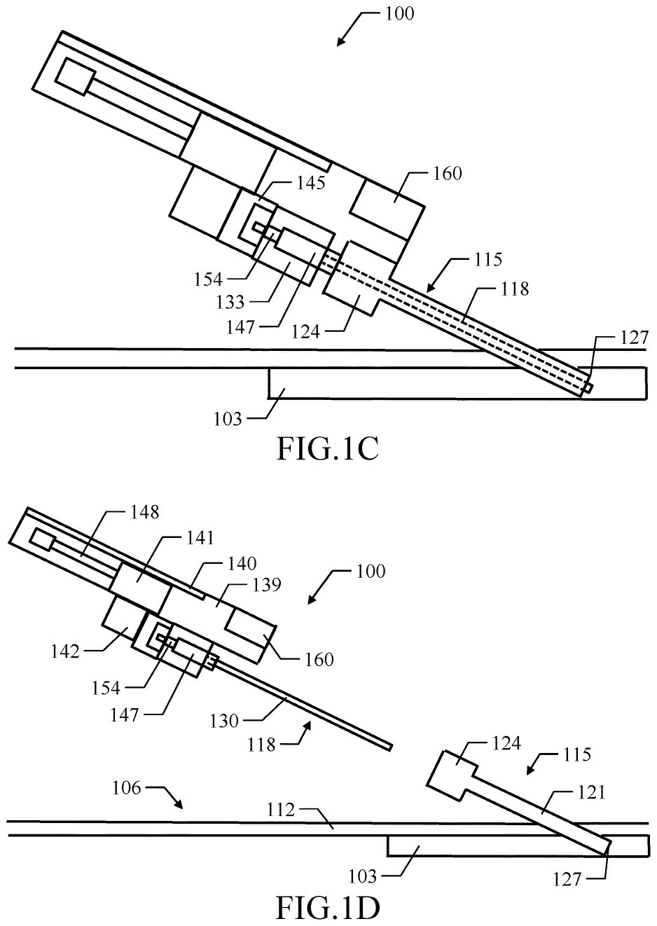

With reference now to FIG. 1A-FIG. 1D, a schematic representation is shown of operation of a catheterization device.

Starting from FIG. 1A, a catheterization device 100 is used for performing peripheral catheterization procedures (in a semi-automatic or completely-automatic way). In this case, the catheterization device 100 allows providing direct access to a target region 103 placed shallowly within a body-part 106 of a patient in each (peripheral) catheterization procedure. The target region 103 has one or more physical characteristics that distinguish it from a rest of the body-part 106. For example, the target region 103 is a blood vessel, such as a peripheral vein (usually placed in a hand or arm). The blood vessel 103 is arranged within subcutaneous tissue 109 covered by skin (dermis and epidermis) 112 of the patient. The blood vessel 103 distinguishes from the skin 112 and the subcutaneous tissue 109 according to the physical characteristics of the blood flowing through it (for example, its bioimpedance).

For this purpose, a catheter 115 and a needle 118, made of (sterilized) medical grade materials, are used. Particularly, the catheter 115 is inserted into the blood vessel 103 via the needle 118 that serves to penetrate the body-part 106 carrying the catheter 115 with it. The catheter 115, for example, a Peripheral Intra Venous Catheter (PIVC), comprises a thin hollow cannula 121 (generally of flexible type), which extends from a larger connector 124 and ends with a mouth 127. The needle 118 comprises a thin rigid shaft 130, which extends from a larger connector 133 and ends with a sharp tip 136. The needle 118 is mounted on the catheterization device 100 in an exchangeable manner (to allow exchanging the needle 118 at the end of the catheterization procedure). In turn, the catheter 115 is mounted on the needle 118 in a releasable manner (to allow releasing the catheter 115 during the catheterization procedure). The tip 136 of the needle 118 projects outside the catheter 115 through its mouth 127 (to allow using the needle 118 to penetrate the body-part 106).

The catheterization device 100 comprises the following components. A bearing structure 139 sustains the other components of the catheterization device 100. A linear guide 140 is arranged on the bearing structure 139 for guiding a slide 141 (for example, with a length of 5-10 cm). A support element 142 is arranged on the slide 141 for supporting a (needle/catheter) assembly comprising the needle 118 and the catheter 115 being mounted thereon. For this purpose, the support element 142 has a connector 145 mating the connector 133 of the needle 118 for mounting it on the catheterization device 100 (in an exchangeable manner). The catheter 115 is mounted on the needle 118 in a relatively loose way (to facilitate its releasing), whereas the needle 118 is mounted on the catheterization device 100 in a relatively firm way (to avoid releasing it as well with the catheter 115). Particularly, a force required to release the catheter 115 from the needle 118 is significantly lower than a force (along the same direction) required to release the needle 118 from the catheterization device 100 (for example, lower than 0.1-0.5 times). For example, the needle 118 is inserted into the catheter 115, with the shaft 130 sliding along the connector 124 and then the cannula 121 until the connector 133 reaches the connector 124 and it is press-fitted into it with relatively low interference. The needle 118 is mounted on the catheterization device 100 with the connector 133 press-fitted onto the connector 145 with relatively high interference or in a way requiring acting along a different direction, such as screwed.

A release element 147 (for example, arranged on the slide 141) is used to release the catheter 115 from the needle 118 (i.e., the connector 124 from the connector 133). A (first) actuator 148 (for example, arranged on the bearing structure 139) moves the slide 141 (and then the support element 142 with the catheter/needle assembly 115,118 integral thereto) along the linear guide 140. A (second) actuator 154 (for example, arranged on the slide 141) moves the release element 147 with respect to the support element 142. A sensor 157 is coupled with the needle 118 for measuring a characteristic signal that is indicative of the physical characteristic (distinguishing the blood vessel 103 from the rest of the body-part 106) of a material contacting the tip 136 of the needle 118. In the example at issue, the sensor 157 measures a bioimpedance. For this purpose, the needle 118 may be of bipolar type with two concentric electrodes. The sensor 157 applies a voltage between these electrodes and measures a corresponding current, from which it calculates the electrical impedance of the material interposed between the electrodes. A control unit 160 controls operation of the catheterization device 100. For example, the control unit 160 comprises (not shown in the figure) a microprocessor, or more, providing a logic capability of the control unit 160, a non-volatile memory (such as a ROM) storing basic code for a bootstrap of the control unit 160, a volatile memory (such as a RAM) used as a working memory by the microprocessor, a mass-memory (such as a Flash E²PROM) storing programs and data and one or more controllers for corresponding peripherals, comprising the actuators 148,154 and the sensor 157.

Moving to FIG. 1B, in operation the catheterization device 100 is placed (manually, semi-automatically or automatically) in correspondence to the blood vessel 103, outside the body-part 106; particularly, in this case the catheterization device 100 is placed to have the needle 118 aligned with the blood vessel 103. In response to a start command imparted by a practitioner, the control unit 160 starts the actuator 148 to move the slide 141 along the linear guide 140. As a result, the support element 142 arranged on the slide 141, and then the catheter/needle assembly 115,118 mounted on the support element 1421, translate so as to advance towards the blood vessel 103. Particularly, the tip 136 of the needle 118 perforates the skin 112 of the patient; the needle 118 then penetrates the body-part 106 (crossing the skin 112), carrying the catheter 115 with it. The sensor 157 continually measures the characteristic signal (indicative of the physical characteristic of the material contacting the tip 136 of the needle 118) and transmits it to the control unit 160. In response to the detection of the reaching of the blood vessel 103 by the tip 136 of the needle 118 (as indicated by the characteristic signal indicative of the blood), the control unit 160 stops the actuator 148.

Moving to FIG. 1C, the control unit 160 then starts the actuator 154 to move the release element 147. The release element 147 applies a force to the catheter 115 that moves it away from the needle 118. The force has an intensity higher than the one required to release the catheter 115 from the needle 118, but lower than the one required to release the needle 118 from the catheterization device 100 (for example, equal to 2-3 times the force required to release the catheter 115 from the needle 118 when the force required to release the needle 118 from the catheterization device 100 is 10 times the force required to release the catheter 115 from the needle 118). As a result, the catheter 115 releases from the needle 118, with the needle 118 that remains mounted on the catheterization device 100. Particularly, the release element 147 acts on the connector 124 of the catheter 115 so as to unfit it from the connector 133 of the needle 118, with the catheter 115 that then slides along the needle 118 (with its mouth 127 still inside the blood vessel 103), whereas the connector 133 of the needle 118 remains press-fitted on the connector 145.

Moving to FIG. 1D, the bearing structure 139 is retracted (manually or automatically), carrying the needle 118 with it. As a result, the needle 118 slips freely along the catheter 115, up to when the shaft 130 of the needle 118 completely leaves the catheter 115. Therefore, the catheter 115 remains in place with its mouth 127 inside the blood vessel 103 and its connector 124 outside the body-part 106 (which connector 124 may then be fixed to the skin 112 by the practitioner, such as with a tape, not shown in the figure), whereas the catheterization device 100 (with the needle 118) is completely removed (with the control unit 160 that, not shown in the figure, activates the actuator 148 to move the slide 141 along the linear guide 140 in the opposite direction so as to retract the support element 142 and activates the actuator 154 to move the release element 147 in the opposite direction so as to retract it—for a next catheterization operation). The catheter 115 (defining the element being functional to the catheterization procedure) may then be exploited as usual to access the blood vessel 103 for performing a variety of medical procedures. For example, the practitioner may connect to the connector 124 of the catheter 115 for administering substances (such as contrast agents, drugs, nutrition and so on), sampling substances (such as blood), draining substances (such as pus), inserting medical instruments (such as a probe or an interventional catheter), measuring pressure (such as intracranial pressure) and so on.

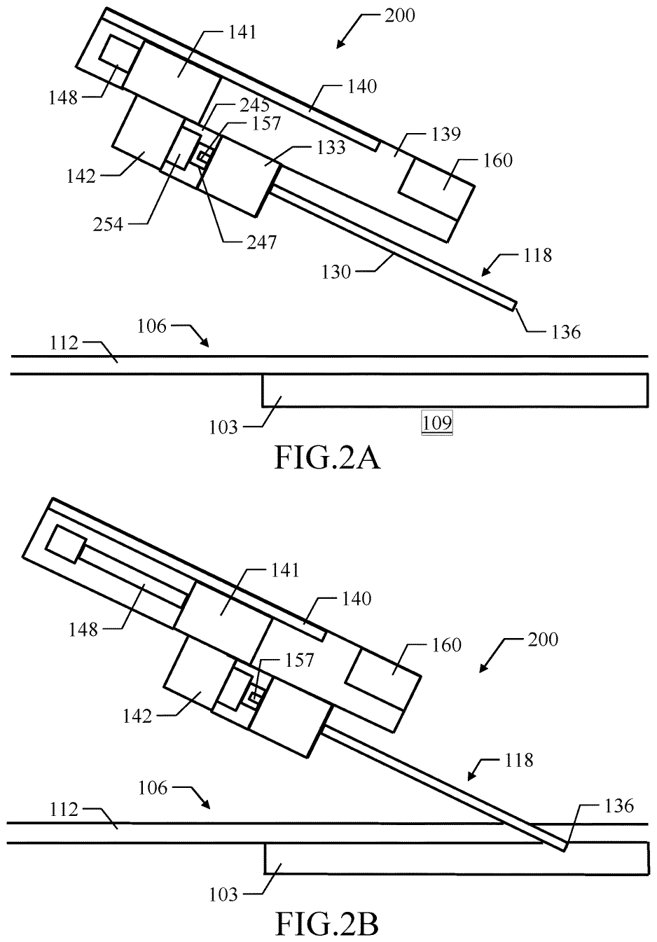

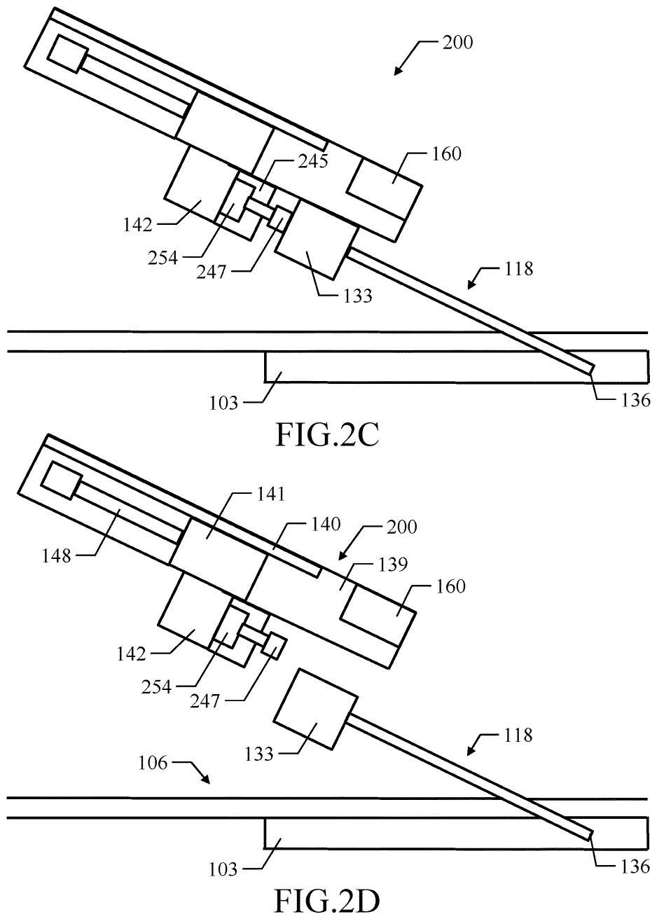

With reference now to FIG. 2A-FIG. 2D, a schematic representation is shown of operation of another catheterization device.

Starting from FIG. 2A, a catheterization device 200 is used for performing deep catheterization procedures (in a semi-automatic or completely-automatic way). In this case, the catheterization device 200 provides direct access to the target region 103 (having one or more physical characteristics that distinguish it from a rest of the body-part 106) for acting more deeply on the patient in each (deep) catheterization procedure. For example, the target region 103 is again a blood vessel, such as a peripheral/central artery (like the femoral, radial or brachial artery) or a central vein (like the internal jugular vein of the neck or the subclavian vein of the chest), arranged within the subcutaneous tissue 109 covered by the skin 112 of the patient. For this purpose, only the needle 118 (typically of bipolar type, with its shaft 130, connector 133 and tip 136) is used in this case to penetrate the body-part 106.

The catheterization device 200 differs from the one described above as follows. The needle 118 is mounted on the catheterization device 200 in a releasable manner. For this purpose, the needle 118 is mounted on the catheterization device 200 in a relatively loose way (to facilitate its releasing during the catheterization procedure). For example, the connector 133 of the needle 118 is press-fitted with relatively low interference onto another connector 245 of the support element 142. The actuator 148 moves the slide 141 (and then the support element 142 now only with the needle 118 integral thereto) along the linear guide 140 (arranged on the bearing structure 139). Another release element 247 (for example, again arranged on the slide 141) is used to release the needle 118 from the catheterization device 200 (i.e., the connector 133 from the connector 245). Another actuator 254 (for example, again arranged on the slide 141) moves the release element 247 with respect to the support element 142. In this case as well, the sensor 157 is coupled with the needle 118 for measuring the same characteristic signal and the control unit 160 controls operation of the catheterization device 200.

Moving to FIG. 2B, in operation the catheterization device 200 is placed in correspondence to the blood vessel 103 (particularly, to have the needle 118 substantially aligned with the blood vessel 103) as above. In response to the start command imparted by the practitioner, the control unit 160 starts the actuator 148 to move the slide 141 along the linear guide 140. As a result, the support element 142 arranged on the slide 141, and then the needle 118 mounted on the support element 142, translate so as to advance towards the blood vessel 103, with the tip 136 of the needle 118 that perforates the skin 112 of the patient and penetrates the body-part 106. As above, the sensor 157 continually measures the characteristic signal and transmits it to the control unit 160; in response to the detection of the reaching of the blood vessel 103 by the tip 136 of the needle 118 (as indicated by the characteristic signal indicative of the blood provided by the sensor 157), the control unit 160 stops the actuator 148.

Moving to FIG. 2C, the control unit 160 then starts the actuator 254 to move the release element 247. The release element 247 applies a force to the needle 118 that moves it away from the support element 142. The applied force has an intensity higher than the one required to release the needle 118 from the catheterization device 200 (for example, equal to 2-3 times it). As a result, the needle 118 is released from the catheterization device 200. Particularly, the release element 247 acts on the connector 133 of the needle 118 so as to unfit it from the connector 245 (with its tip 136 still inside the blood vessel 103).

Moving to FIG. 2D, the bearing structure 139 is retracted (manually or automatically) leaving the needle 118 in place with the tip 136 inside the blood vessel 103 and the connector 133 outside the body-part 106, whereas the catheterization device 200 is completely removed (with the control unit 160 that, not shown in the figure, activates the actuator 148 to move the slide 141 along the linear guide 140 in the opposite direction so as to retract the support element 142 and activates the actuator 254 to move the release element 247 in the opposite direction so as to retract it-for a next catheterization operation). The needle 118 (defining the element being functional to the catheterization procedure) may then be exploited as usual to access the blood vessel 103 for performing a variety of medical procedures more deeply on the patient. For example, the practitioner may insert a guidewire through the needle 118 up to reach a desired target organ (such the heart), remove the needle 118, insert a guiding catheter (for example, a Central Venous Catheter (CVC)) and a dilator over and along the guidewire, remove the guidewire and the dilator together, and then insert a suitable instrument into the guiding catheter (such as an interventional or diagnostic catheter for intervention/diagnosis of the target organ).

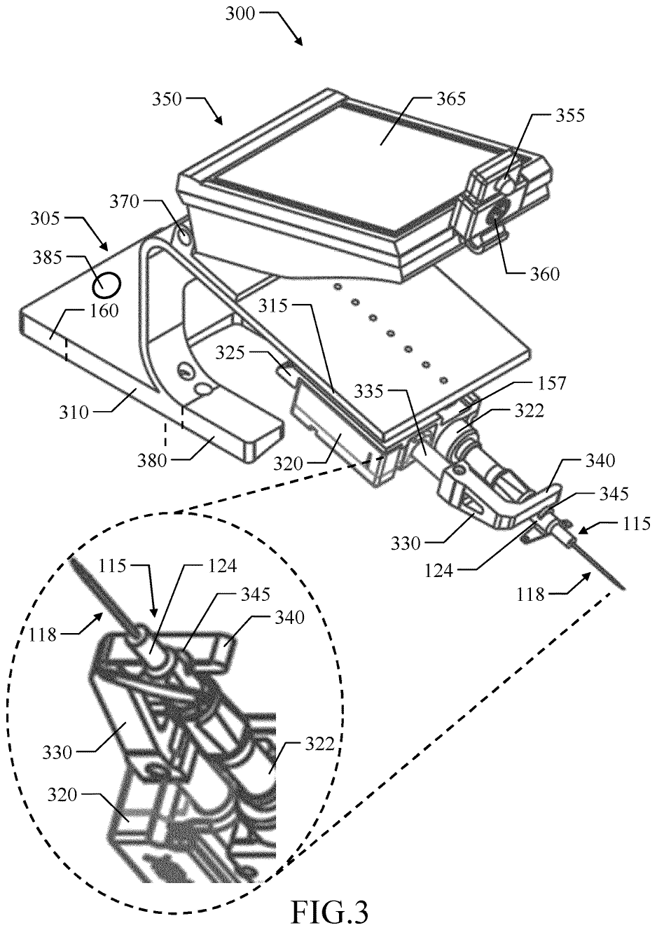

With reference now to FIG. 3, a pictorial representation is shown of a catheterization device 300 of hand-held type. For the sake of simplicity, reference is made to the peripheral catheterization procedure only (however, with the same considerations that apply to the deep catheterization procedure as well).

The catheterization device 300 is configured to be operated by hand by a practitioner. For this purpose, the catheterization device 300 comprises the following components. The bearing structure (for example, implemented by a chassis), differentiated with the reference 305, is sized so as to be held by a single hand of the practitioner. The bearing structure 305 comprises a foot 310 (for example, with a bottom flat surface) for resting the catheterization device 300 onto the skin of the patient (not shown in the figure) during the catheterization procedure. The bearing structure 305 comprises the linear guide (for example, implemented by a track), differentiated with the reference 315, for guiding the slider (for example, implemented by a carriage), differentiated with the reference 320, on which the support element for the catheter/needle assembly 115,118 (for example, implemented by a base), differentiated with the reference 322, is arranged. The corresponding actuator (for example, implemented by a linear motor), differentiated with the reference 325, is mounted on the bearing structure 305 for translating the slide 320 along the linear guide 315. The linear guide 315 is sloped with respect to the foot 310; for example, the linear guide 315 forms with the bottom surface of the foot 310 an angle of 30-40°, preferably 32-38° and still more preferably 34-36°, such as 35°. In this way, the needle 118 is inserted into the body-part with an angle that facilitates its penetration. The release element (for example, implemented by a finger), differentiated with the reference 330, extends from the slide 320 to the connector 124 of the catheter 115 at a side of the needle 118 (the release element 330 extends substantially parallel to the catheter/needle assembly 115,118 and slightly spaced apart therefrom). The corresponding actuator (for example, implemented by a linear motor), differentiated with the reference 335, is mounted on the slide 320 for translating the release element 330 with respect thereto. The release element 330 ends (at an opposite side of the actuator 335) with a nail 340 that is bent by 90° so as to extend transversally to the catheter 115. The nail 340 and the connector 124 of the catheter 115 have matching features allowing the nail 340 to act on the connector 124 without interfering with the needle 118; for example, the nail 340 is configured to push a protrusion of the connector 124 and is provided with a recess 345 for sliding along the needle 118. The release element 330 is mounted on the slide 320 in a removable manner (for example, screwed thereon), so that it may be replaced in a relatively easy way; this allows changing the release element 330 to adapt the catheterization device 300 to different structures of the catheter 115.

A guidance module 350 is used to facilitate the task of the practitioner in identifying the blood vessel that is best suited for the catheterization procedure and in positioning the catheterization device 300 correctly with the needle 118 substantially aligned with the blood vessel (not shown in the figure). The guidance module 350 comprises the following components. An acquisition unit (comprising a light source 355 and a camera 360) is used to acquire images representative of an interior of the body-part in front of the catheterization device 300 (where the needle 118 is to be inserted). The light source 355 (for example, based on one or more LEDs) illuminates the body-part with a Near InfraRed (NIR) light (i.e., with a wavelength just above the visible spectrum, such as in the range from 700 nm to 3,500 nm). For example, the light source 355 generates NIR light at 900-1,000 nm (such as 950 nm); this provides a good level of penetration inside the body-part (such as to a depth of 3-4 mm) and a good quality of its representation for different types of tone of the skin of the patient. The camera 360 acquires NIR images of the same body-part. For example, the camera 360 has optical lens keeping its focal length at 7-15 cm (such as 10 cm) and sensors (such as of CCD type) for the NIR light reflected from the body-part. As a result, the NIR images represent the body-part substantially without colors but with improved visibility of internal details thereof. The control unit 160, for example, via a dedicated Graphics Processing Unit (GPU), applies image processing techniques to the NIR images provided by the camera 360 to enhance the representation of any blood vessels therein. The control unit 160 generates corresponding (enhanced) NIR images of the body-part, which provide an augmented-reality representation thereof (wherein a real structure of the body-part is complemented by computer-generated information relating thereto). A monitor 365 displays the enhanced NIR images of the body-part to the practitioner. The guidance module 350 is mounted on the bearing structure 305 via a hinge 370, which extends transversally to the linear guide 315. In this way, the guidance module 350 may pivot around the hinge 370, between an unfolded condition (as shown in the figure) for facilitating the practitioner in looking at the monitor 365, and a folded condition (not shown in the figure) for limiting its size when the catheterization device 300 is not in use. The guidance module 350 may be easily attached to and detached from the bearing structure 305 (for example, by means of a snap-fitting mechanism). In this way, when the blood vessel is clearly visible to the naked eye the catheterization device 300 may be used in a simplified version without the guidance module 350, whereas when the blood vessel is difficult to see to the naked eye the catheterization device 300 may be used in a complete version with the guidance module 350.

The bearing structure 305 houses a battery 380 (for example, of rechargeable type), which supplies all the active components of the catheterization device 300, comprising the actuators 325 and 335, the (possible) guidance module 350, the sensor 157 and the control unit 160. The bearing structure 305 is also provided with a manual command 385 (for example, a push-button) that is used by the practitioner to start each catheterization procedure.

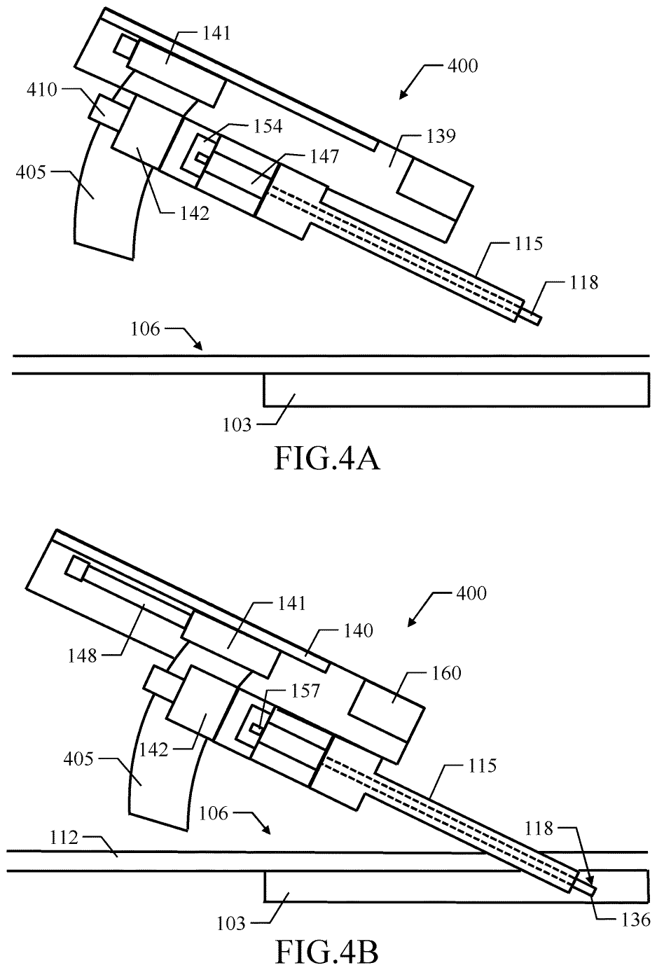

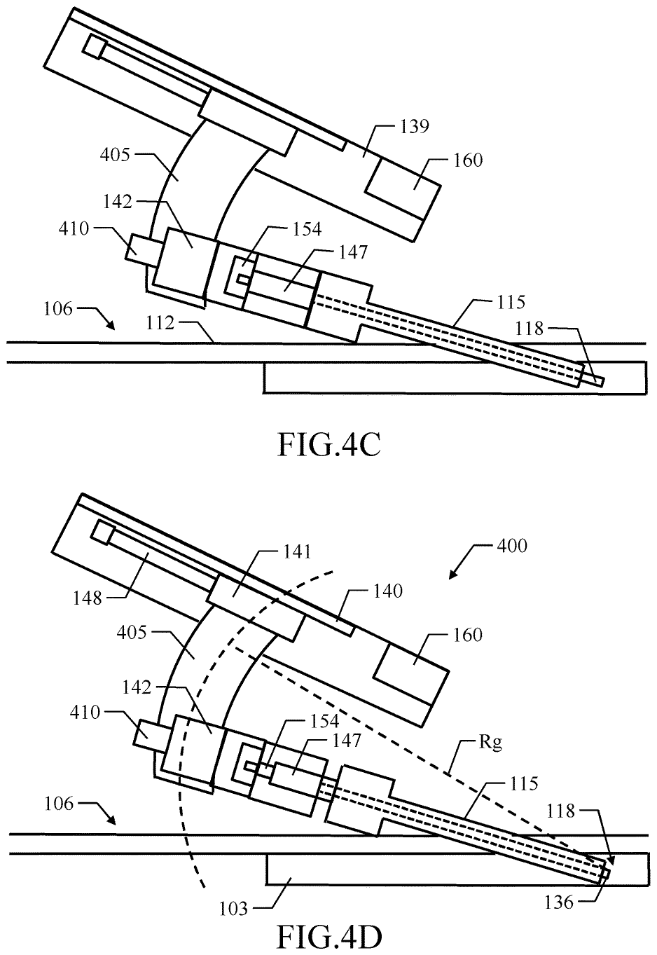

With reference now to FIG. 4A-FIG. 4D, a schematic representation is shown of the principles of the solution according to an embodiment of the present disclosure. For the sake of simplicity, reference is made to the peripheral catheterization procedure only (however, with the same considerations that apply to the deep catheterization procedure as well).

Starting from FIG. 4A, the catheterization device, differentiated with the reference 400, differs from the one of FIG. 1A-FIG. 1D as follows. Particularly, the catheterization device 400 comprises a curved guide 405 (for example, extending along an arc, such as with a length of 3-10 cm, of a circumference, such as with a radius of 20-50 cm). The curved guide 405 is arranged on the slide 141 for guiding the support element 142 (and preferably the release element 147 and the actuator 154 as well). A (third) actuator 410, arranged on the bearing structure 139 or on the slide 141, moves the support element 142 (and then the catheter/needle assembly 115,118 integral thereto) along the curved guide 405.

Moving to FIG. 4B, in operation the catheterization device 400 is placed in correspondence to the blood vessel 103, outside the body-part 106 (particularly, to have the needle 118 aligned with the blood vessel 103) as above. In response to the start command imparted by the practitioner, the control unit 160 starts the actuator 148 to move the slide 141 along the linear guide 140. As a result, the support element 142 arranged on the curved guide 405 in turn on the slide 141, and then the catheter/needle assembly 115,118 mounted on the support element 142, translate so as to advance towards the blood vessel 103, with the tip 136 of the needle 118 that perforates the skin 112 of the patient and penetrates the body-part 106. As above, the sensor 157 continually measures the characteristic signal and transmits it to the control unit 160; in response to the detection of the reaching of the blood vessel 103 by the tip 136 of the needle 118 (as indicated by the characteristic signal indicative of the blood provided by the sensor 157), the control unit 160 stops the actuator 148.

Moving to FIG. 4C, the control unit 160 starts the actuator 410 to move the support element 142 (and preferably the release element 147 and the actuator 154 as well) along the curved guide 405. As a result, the support element 142 (and possibly the release element 147 and the actuator 154) rotates with respect to the bearing structure 139. This causes the catheter/needle assembly 115,118 (mounted on the support element 142) to rotate accordingly, thereby reducing an angle between the catheter/needle assembly 115,118 and the body part 106 (i.e., the skin 112).

Moving to FIG. 4D, as above the control unit 160 then starts the actuator 154 to move the release element 147, so as to release the catheter 115 from the needle 118 (with the needle 118 that remains mounted on the catheterization device 400). The catheterization device 400 is then removed (not shown in figure) carrying the needle 118, whereas the catheter 115 remains in place (with the control unit 160 that, not shown in the figure, activates the actuator 148 to move the slide 141 along the linear guide 140 in the opposite direction so as to retract the support element 142, activates the actuator 410 to move the support element 142 along the curved guide 405 in the opposite direction so as to rotate it back and activates the actuator 154 to move the release element 147 in the opposite direction so as to retract it—for a next catheterization operation).

The above-described solution automates the most critical and difficult parts of the catheterization procedure, i.e., the penetration of the body part by the needle, the detection of the reaching of the blood vessel, the rotation of the needle after its insertion into the body-part and the release of the functional element (catheter or needle). Particularly, the (autonomous) insertion of the needle into the body-part makes the operation safer and more precise. The (autonomous) detection of the reaching of the blood vessel avoids (or at least substantially reduces) the risk of too shallow or too deep insertion of the needle (and then of not accessing the blood vessel or of crossing the blood vessel, respectively). The (autonomous) rotation of the needle allows inserting the needle into the body-part with a higher angle (facilitating its penetration) and at the same time releasing the functional element at a lower angle (thereby facilitating access to the blood vessel thanks to a correct and safe positioning of the functional element along a direction substantially parallel to the longitudinal axis of the blood vessel). The (autonomous) release of the functional element avoids (or at least substantially reduces) the risk of moving the functional element from its correct position in the blood vessel.

Particularly, the curved guide being mounted on the slide ensures that the rotation of the needle always occurs along an optimized rotation path defined by the curved guide, irrespectively of the translation of the needle required for its insertion into the body-part. In fact, the translation of the slide along the linear guide moves the needle, and then its axis of rotation accordingly; however, this does not affect the rotation path of the needle, since it is defined by the curved guide that moves with the needle in the same way.

All of the above significantly reduces a failure rate of the catheterization procedure. Therefore, in most cases the functional element may be inserted successfully with a single attempt (even in case of patients with medical situations leading to difficult access to the blood vessel); this is especially useful in case of patients that are very young (children), very old, with dark complexion or with chronic conditions (such as obesity, diabetes, parenteral drug abuse, undergoing chemotherapy and so on). The obtained result is substantially independent of the skills (and attention) of the practitioner, and it is then less prone to human errors and highly reproducible. As a result, it is possible to reduce unwanted consequences for the patients, with a beneficial effect on their health.

Particularly, in a preferred implementation the curved guide 405 has a curvature radius Rg equal to a distance of the tip 136 of the needle 118 (mounted on the support element 142) from the curved guide 405 (as defined by a length of an arm defined by the part of the support element 142 with the needle 118 that projects inwards the curved guide 405). In this way, the needle 118 always rotates around an axis of rotation (perpendicular to a longitudinal axis thereof) that passes through the tip 136 of the needle 118 irrespectively of its position (not known a priori, since depending on the translation of the slide 141 for inserting the needle 118 into the body-part 106 according to a depth of the blood vessel 103 within the body-part 106).

This avoids (or at least significantly reduces) any risk of damages to the patient, notwithstanding the rotation of the needle 118 occurs when it is already inside the blood vessel 103.

Advantageously, the support element 142 may be adjusted according to a length of the needle 118, so as to set the distance of the tip 136 of the needle 118 from the curved guide 405 to match the curvature radius Rg of the curved guide 405. This allows maintaining the axis of rotation of the needle 118 passing through its tip 136 for different lengths of the needle 118. For example, the support element 142 has a telescopic structure, with two concentric tubular sections designed to slide each into the other (so as to change their length) and a locking mechanism (such as a cap screw) for fixing them in position. In this way, the catheterization device 400 may be adapted to different types of needle 118 in a very simple way.

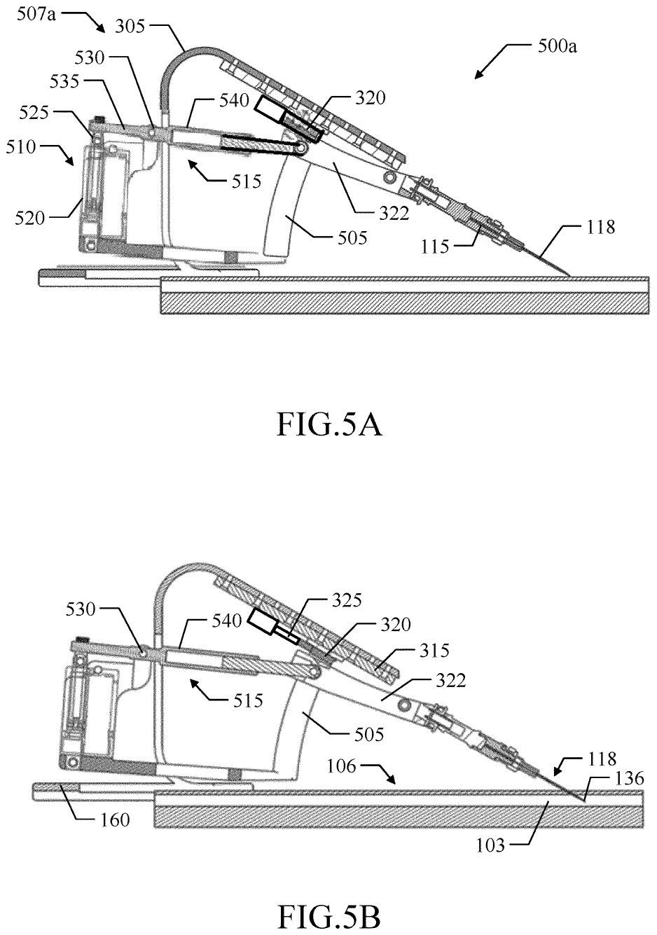

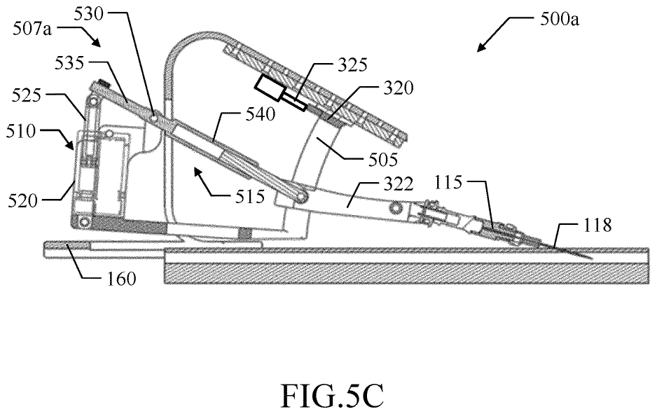

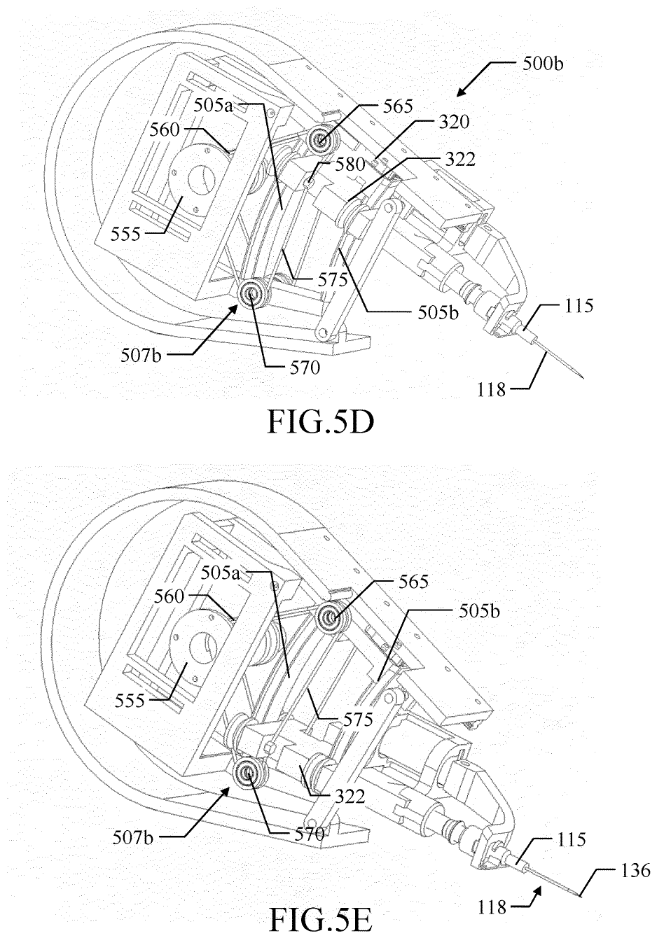

With reference now to FIG. 5A-FIG. 5C and FIG. 5D-FIG. 5E, operation is shown of different catheterization devices of hand-held type according to an embodiment of the present disclosure. For the sake of simplicity, reference is made to the peripheral catheterization procedure only (however, with the same considerations that apply to the deep catheterization procedure as well).

Particularly, an implementation of the catheterization device is shown in FIG. 5A-FIG. 5C.

Starting from FIG. 5A, the catheterization device, differentiated with the reference 500a, differs from the one described above (with reference to FIG. 3) as follows. Particularly, the catheterization device 500a comprises the curved guide (for example, implemented by a curved track), differentiated with the reference 505, that is integral with the slide 320. The actuator for rotating the support element 322, differentiated with the reference 507a, is arranged on the bearing structure 305. The actuator 507a comprises a cylinder 510 and a lever 515. Particularly, the cylinder 510 comprises a barrel 520 (integral with the bearing structure 305) and a piston 525 (slidable along the barrel 520). The lever 515 has a (fixed) fulcrum 530 on the bearing structure 305. The fulcrum 530 divides the lever 515 into an effort arm 535 (for receiving an input force) and a load arm 540 (for applying an output force). The effort arm 535 has a fixed length, whereas the load arm 540 has a variable length (for example, with a telescopic structure defined by two concentric tubular sections that are free to slide each into the other). The piston 525 is hinged to the effort arm 535 (between a free end of the piston 525 projecting from the barrel 520 and a free end of the effort arm 535 distal from the fulcrum 530) and the load arm 540 is hinged to the support element 322 (between a free end of the load arm 540 distal from the fulcrum 530 and a free end of the support element 322 opposite the catheter/needle assembly 115,118).

Moving to FIG. 5B, as above the actuator 325 moves the slide 320 along the linear guide 315. As a result, the support element 322 arranged on the curved guide 505 in turn on the slide 320, and then the catheter/needle assembly 115,118 mounted on the support element 322, translate so as to advance towards the blood vessel 103, with the tip 136 of the needle 118 that penetrates the body-part 106 until the control unit 160 detects that the tip 136 of the needle 118 has reached the blood vessel 103. In this phase, the load arm 540 of the lever 515 slightly lengthens (according to the moving away of the slide 320 being hinged thereto from the fulcrum 530).

Moving to FIG. 5C, once the control unit 160 has stopped the actuator 325 (but before releasing the catheter 115 from the needle 118), the control unit 160 starts the actuator 507a. Particularly, the cylinder 510 is activated so that the piston 525 is extracted from the barrel 520. The piston 525 then applies an input force to the effort arm 535 (hinged thereto) that causes the lever 515 to rotate around the fulcrum 530 (clockwise in the figure). Consequently, the corresponding output force applied by the load arm 540 to the support element 322 (hinged thereto) moves it along the curved guide 505 (downwards in the figure), thereby causing the support element 322 (and then the catheter/needle assembly 115,118 mounted thereon) to rotate in the opposite direction (counterclockwise in the figure). In this phase, the load arm 540 of the lever 515 further lengthens (according to the moving away of the slide 320 being hinged thereto from the fulcrum 530).

Another implementation of the catheterization device is instead shown in FIG. 5D-FIG. 5E.

Starting from FIG. 5D, the catheterization device, differentiated with the reference 500b, now differs from the one described above (with reference to FIG. 3) as follows. Particularly, the catheterization device 500b has the actuator for rotating the support element 322, differentiated with the reference 507b, that is instead arranged on the slide 320. Moreover, preferably the curved guide is formed by two curved elements 505a and 505b (for example, corresponding rails) that are concentric. The curved element 505b is arranged inside the curved element 505a, and accordingly it is shorter and with a smaller curvature radius (for example, equal to ⅓-¼). The actuator 507b comprises the following components. A rotational motor 555 rotates a driving pulley 560 around an axis of rotation (of a corresponding motor shaft) being parallel to the one of the support element 322 (for example, outside the curved element 505a). One or more idler pulleys, two in the example at issue, denoted with the references 565 and 570, rotate freely around corresponding axes of rotations (of corresponding axles) parallel to the one of the support element 322 (for example, at the ends of the curved element 505a). A looped transmission element, for example, a belt 575 is fitted onto the (motor/idler) pulleys 560-570. A coupler 580 (for example, a screw) couples the support element 322 with the belt 575. Particularly, the figure shows the support element 322 in a (starting) position for insertion of the catheter/needle assembly 115,118 into the body-part of the patient (not shown in the figure).

Moving to FIG. 5E, as above once the tip 136 of the needle 118 has reached the blood vessel (not shown in the figure) but before releasing the catheter 115 from the needle 118, the actuator 507b is activated. Particularly, the rotational motor 555 is started so as to rotate the driving pulley 560. The rotation of the driving pulley 560 is transmitted to the belt 575 (with the idler pulleys 565-570 that are made to rotate accordingly). The rotation of the belt 575 (clockwise in the figure) pulls the support element 322 (integral therewith) so as to move along the curved guide 505a,505b accordingly (downwards in the figure), thereby rotating the support element 322 (and then the catheter/needle assembly 115,118 mounted thereon) in the opposite direction (counterclockwise in the figure).

Both the above-described implementations of the actuator allow rotating the needle smoothly. Moreover, this result is achieved with a structure that is relatively compact (especially well-suited for a catheterization device of hand-held type).

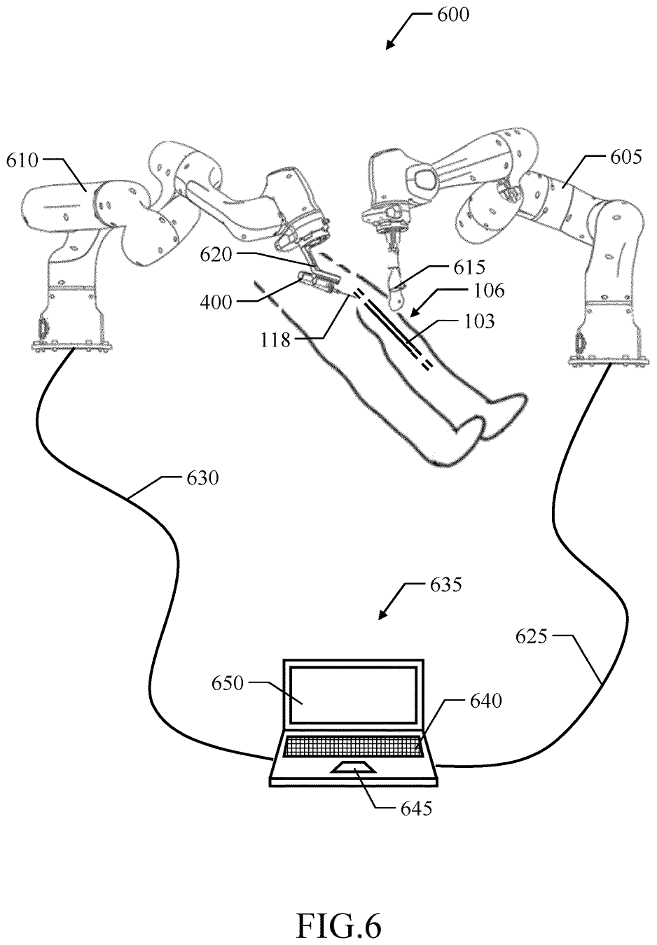

With reference now to FIG. 6, a pictorial representation is shown of a robotic system 600 comprising a catheterization device according to an embodiment of the present disclosure. For the sake of simplicity, reference is made to the deep catheterization procedure only (however, with the same considerations that apply to the peripheral catheterization procedure as well).

The robotic system 600 is used to perform deep catheterization procedures in an automatic (or at least semi-automatic) way. The robotic system 600 comprises the following components. Two (independent) robotic arms 605 and 610 are formed each by an articulated chain of links connected by joints that are moved by motors (not visible in the figure); the articulated chain provides 7 (or more) degrees of freedom to an end thereof, so as to allow it to reach any arbitrary pose (position and orientation) in three dimensional space. The robotic arm 605 carries an ultrasound probe 615 at its free end. The robotic arm 610 instead carries a holder 620 at its free end. The catheterization device 400 is mounted (in a removable manner) on the holder 620 to define an end-effector of the robotic system 600. Advantageously, the holder 620 has an angled structure, formed by a support that ends with an (inclined) platform forming an angle of 30-50° with respect thereto; the catheterization device 400 is mounted on the inclined platform of the holder 620. This facilitates the correct positioning of the catheterization device 400 for the insertion of the needle 118 into the body-part 106 of the patient. The arms 605 and 610 are coupled, for example, via corresponding wired connections 625 and 630, respectively, to a driving device 635, for example, implemented by a Personal Computer (PC). Particularly, the driving device 635 comprises (not show in the figure) a microprocessor, or more, providing a logic capability of the driving device 635, a non-volatile memory (such as a ROM) storing basic code for a bootstrap of the driving device 635, a volatile memory (such as a RAM) used as a working memory by the microprocessor, a mass-memory (such as an SSD) storing programs and data, and one or more controllers for corresponding peripherals, comprising the robotic arms 605 and 610, the ultrasound probe 615, the catheterization device 400, a keyboard 640, a tracking unit 645 and a monitor 650.

The ultrasound probe 615 is used to detect the blood vessel 103; this information is then used to position the catheterization device 400 accordingly (by the robotic arm 610). The above-described solution automates the whole catheterization procedure (or at least most of it), thereby further reducing its failure rate.

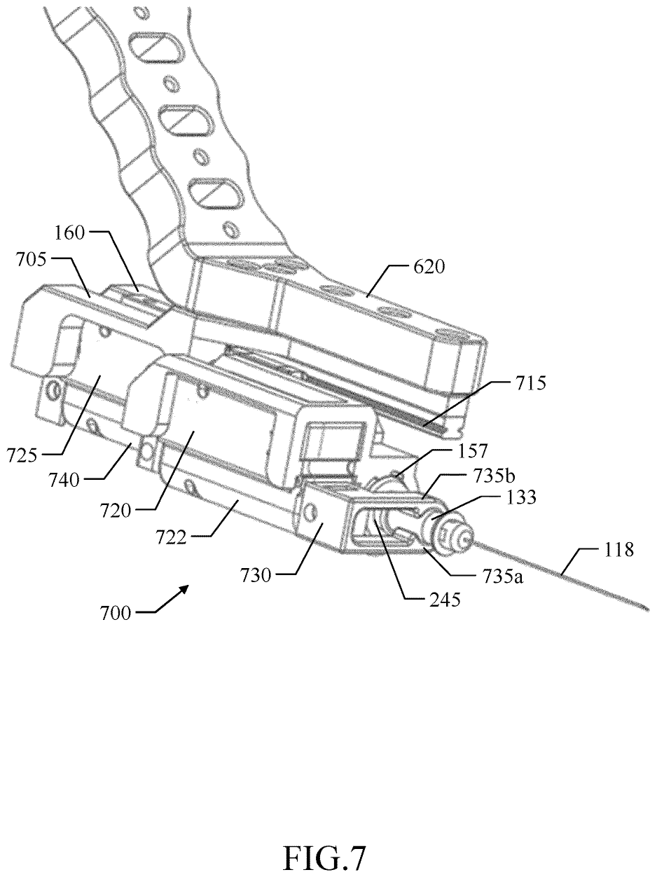

With reference now to FIG. 7, a pictorial representation is shown of a catheterization device of end-effector type. For the sake of simplicity, reference is made to the deep catheterization procedure only (however, with the same considerations that apply to the peripheral catheterization procedure as well).

The catheterization device (for use in the above-described robotic system, only partially shown in the figure), differentiated with the reference 700, comprises the following components. The bearing structure comprising the control unit 160 (for example, implemented by a head), differentiated with the reference 705, is configured to match the holder 620 of the robotic system. The bearing structure 705 has the linear guide (for example, implemented by a track), differentiated with the reference 715, which extends parallel to the holder 620, for the slide (for example, implemented by a carriage), differentiated with the reference 720; the support element for the needle 118 with the connector 245 and the sensor 157 (for example, implemented by a base), differentiated with the reference 722, is arranged on the slide 720. The corresponding actuator (for example, implemented by a linear motor), differentiated with the reference 725, is mounted on the bearing structure 705 for translating the slide 720 along the linear guide 715. The release element (for example, implemented by a clamp), differentiated with the reference 730, extends from the support element 722 to the connector 133 of the needle 118; the release element 730 has two jaws 735a and 735b (movable relative to each other) matching the connector 133 of the needle 118 (for clamping the connector 133 when closed onto it). The corresponding actuator (for example, implemented by a linear motor), differentiated with the reference 740, translates the release element 730 with respect to the support element 722. The release element 730 is mounted on the support element 722 in a removable manner (for example, screwed thereon), so that it may be replaced in a relatively easy way; this allows changing the release element 730 to adapt the catheterization device 700 to different structures of the needle 118.

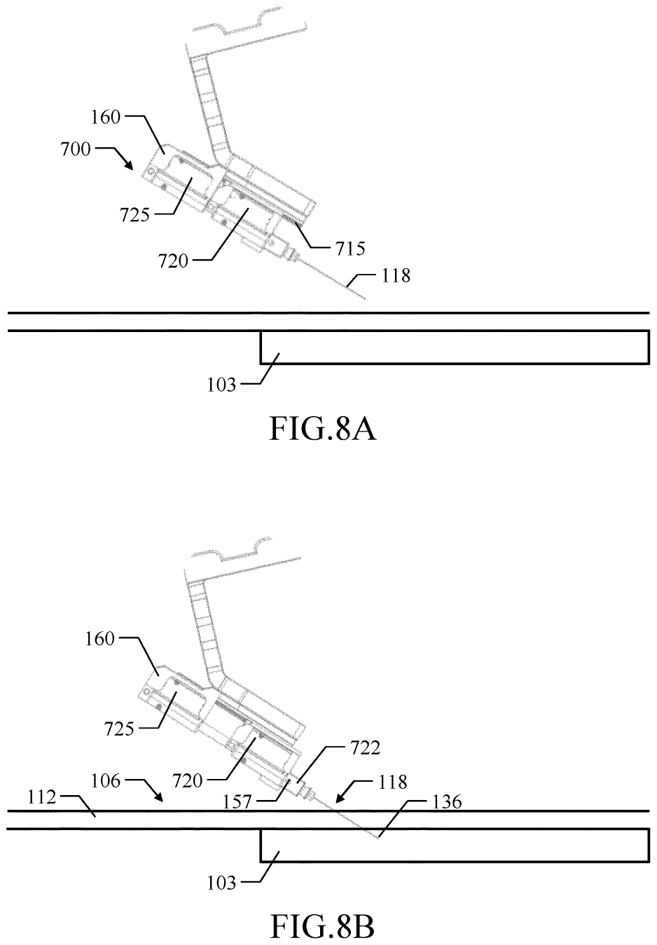

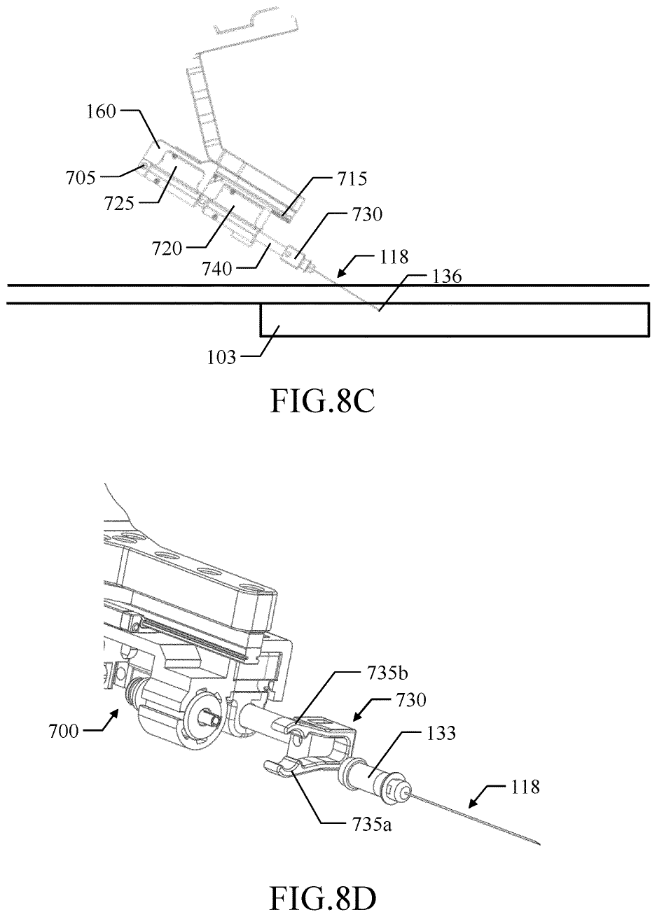

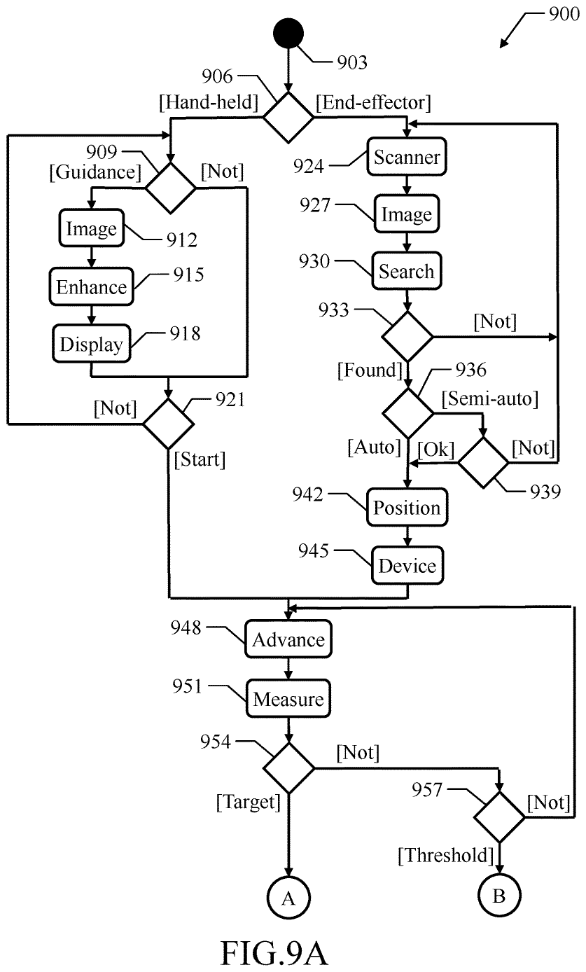

With reference to FIG. 8A-FIG. 8D, operation is shown of this catheterization device 700 of end-effector type.

Starting from FIG. 8A, in operation the catheterization device 700 is placed in correspondence to the blood vessel 103 as above (particularly, to have the needle 118 aligned with it). In response to a start command imparted by the practitioner (via the driving device of the robotic system, not shown in the figure), the control unit 160 starts the actuator 725 to translate the slide 720 forwards along the linear guide 715.

Moving to FIG. 8B, as a result the support element 722 arranged on the slide 720, and then the needle 118 mounted on the support element 722, translate so as to advance towards the blood vessel 103, with the tip 136 of the needle 118 that perforates the skin 112 of the patient and penetrates the body-part 106. As above, the sensor 157 continually measures the characteristic signal and transmits it to the control unit 160; in response to the detection of the reaching of the blood vessel 103 by the tip 136 of the needle 118 (as indicated by the characteristic signal indicative of the blood provided by the sensor 157), the control unit 160 stops the actuator 725.

Moving to FIG. 8C, the control unit 160 then starts the actuator 740 to translate the release element 730 forwards in parallel to the linear guide 715. At the same time, the control unit 160 commands the actuator 725 to translate the slide 720 backwards along the linear guide 715. The actuator 740 and the actuator 725 act in opposite directions at the same speed (in absolute value). The relative movement of the release element 730 with respect to the slide 720 (away from it) releases the needle 118 from the slide 720. At the same time, the relative movement of the needle 118 with respect to the bearing structure 705 is null (thanks to the opposite relative movement of the slide 720 with respect to the linear guide 715 integral with the bearing structure 705). As a result, the needle 118 (and particularly its tip 136 within the blood vessel 103) remains in the same position.

Moving to FIG. 8D, the release element 730 is opened by moving its jaws 735a and 735b away from each other (for example, in an automatic manner or manually by the practitioner), so as to disengage the connector 133 of the needle 118 from the release element 730. The catheterization device 700 may then be removed leaving the needle 118 in place.

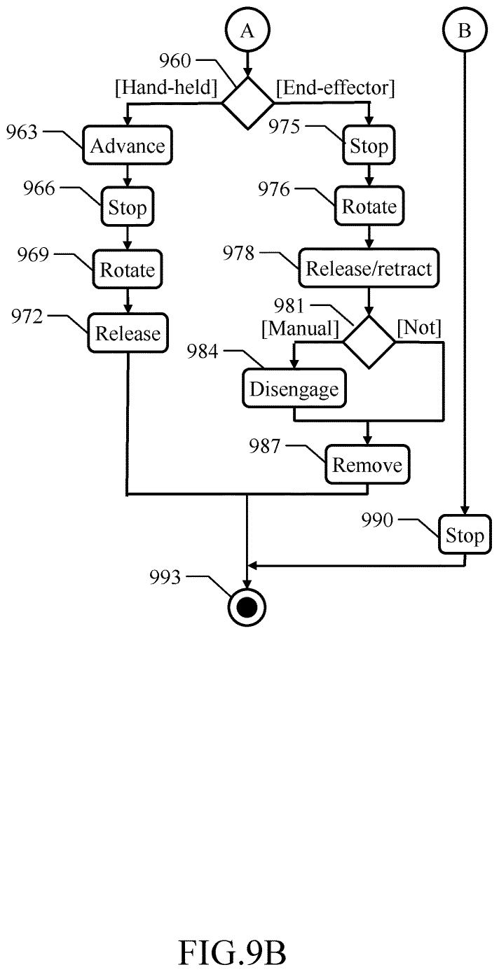

With reference now to FIG. 9A-FIG. 9B, an activity diagram is shown describing the flow of activities relating to an implementation of the solution according to an embodiment of the present disclosure.

Particularly, the diagram represents an exemplary process that may be used to operate the catheterization device (during each catheterization procedure) with a method 900. In this respect, each block may correspond to one or more executable instructions for implementing the specified logical function on the relevant computing machines (i.e., control unit of the catheterization device and possible driving device of the robotic system). The corresponding software components (comprising a control manager running on the control unit and a possible driving manager running on the driving device) are typically stored in the mass memory and loaded (at least partially) into the working memory of the computing machines when the programs are running. The programs are initially installed into the mass memory, for example, from removable storage units or from a network.

The process begins at the black start circle 903 when the catheterization device is switched on, and then branches at block 906 according to a type of the catheterization device (for example, set during a configuration of its control manager). For the sake of simplicity, reference is made to the peripheral catheterization procedure only for the catheterization device of hand-held type and to the deep catheterization procedure only for the catheterization device of end-effector type (however, with similar considerations that apply to the deep catheterization procedure with the catheterization device of hand-held type and to the peripheral catheterization procedure with the catheterization device of end-effector type as well). If the catheterization device is of hand-held type blocks 909-921 are executed, whereas if the catheterization device is of end-effector type blocks 924-945 are executed. In both cases, the process merges again at block 948. Alternatively (not shown in the figure), the program is provided with corresponding versions for the different types of the catheterization device.

With reference now to block 909 (catheterization device of hand-held type), the process branches according to a configuration of the catheterization device. If the catheterization device is provided with the guidance module (for example, as detected automatically or being indicated by a configuration parameter), the control manager at block 912 commands the acquisition unit of the guidance module to acquire a NIR image of its field of view (comprising the body-part with the possible target region of the catheterization procedure after the practitioner has arranged the catheterization device onto it). The control manager at block 915 enhances the (possible) representation of the target region in the NIR image. For example, the control manager identifies the target region with segmentation techniques and then highlights it in color. The control manager at block 918 commands the monitor of the guidance module to display the (enhanced) NIR image. The process then descends into block 921; the same point is also reached directly from 909 if the catheterization device is not provided with the guidance module. In both cases, the control manager now verifies whether the start command has been entered by the practitioner (via the manual command). If not, the process returns to block 909 to repeat the same operations. Conversely, the flow of activity descends into block 948 as soon as the practitioner has entered the start command to confirm that the catheterization device is positioned correctly (as determined either with the aid of the guidance module or to the naked eye).

With reference instead to block 924 (catheterization device of end-effector type), the practitioner uses the driving manager to command the robotic arm carrying the ultrasound probe to position it at the body-part with the possible target region of the catheterization procedure. The driving manager at block 927 then commands the ultrasound probe to acquire an ultrasound image of its field of view (comprising the body-part with the possible target region), at the same time displaying it onto the monitor of the driving device. The driving manager at block 930 searches for the representation of the target region in the ultrasound image. For example, the driving manager identifies the (possible) target region with image recognition techniques. The flow of activity branches at block 933 according to a result of this search. If the target region has not been found, the process returns to block 924 to repeat the same operations (with the practitioner that may move the robotic arm carrying the ultrasound probe to better position it). Conversely, the flow of activity descends into block 936 as soon as the target region has been found. At this point, the process branches according to a configuration of the robotic system (for example, set manually during a configuration thereof, selected dynamically with a possible default value or the only one available). Particularly, if the robotic system is configured to operate in a semi-automatic mode, the driving manager at block 939 prompts the practitioner to confirm the finding of the target region in the ultrasound image (on the driving device). If the practitioner refuses the target region being found, the process returns to block 924 to repeat the same operations. Conversely, the flow of activity descends into block 942 if the practitioner accepts the target region being found; the same point is also reached directly from block 936 if the robotic system is configured to operate in a (completely) automatic mode. At this point, the driving manager determines a (target) position of the catheterization device being required for inserting the needle into the target region correctly, with the needle aligned with it (if necessary) and forming a pre-defined angle with the body-part facilitating its insertion (such as 30-40°) according to the position of the target region in the ultrasound image. The driving manager at block 945 commands the robotic arm carrying the catheterization device to position it accordingly. The process then descends into block 948.

With reference now to block 948, the control manager commands the corresponding actuator to start advancing the slide (with the support element on which the catheter/needle assembly or the needle are mounted) towards the target region. At the same time, the control manager at block 951 commands the sensor to start measuring the corresponding characteristic signal. The flow of activity branches at block 954 according to this characteristic signal. If the characteristic signal is not indicative of the target region, the control manager at block 957 verifies an elapsed time from the start of the actuator. If the elapsed time is lower than a threshold value (corresponding to a maximum stroke of the support element that is allowed, for example, 30-50 mm), the process returns to block 948 to continue advancing the support element. With reference again to block 954, the process passes to block 960 as soon as the characteristic signal is indicative of the target region (with the control manager that possibly commands the sensor to stop measuring the corresponding characteristic signal).

The process now branches again according to the type of the catheterization device. If the catheterization device is of the hand-held type blocks 963-972 are executed, whereas if the catheterization device is of the end-effector type blocks 975-987 are executed.

With reference now to block 963 (catheterization device of hand-held type), the control manager further advances the support element by a pre-defined distance (such as 1-2 mm); this ensures that the mouth of the catheter as well has reached the target region. The control manager at block 966 then stops the actuator. The control manager at block 969 commands the corresponding actuator to rotate the support element (with the catheter/needle assembly). For example, the support element is rotated by 5-15°, preferably 7-13° and still more preferably 9-11°, such as 10°, so as to bring the catheter/needle assembly to be almost parallel to the skin of the patient (such as forming an angle with it of about 0-10°). The control manager at block 972 commands the corresponding actuator to advance the release element (so as to release the catheter from the needle). Particularly, the actuator advances the release element for a distance that is sufficient to release the catheter from the needle while ensuring that its mouth remains within the target region, for example, 0.5-1.5 mm, preferably 0.7-1.3 mm and still more preferably 0.9-1.1 mm, such as 1.0 mm. At this point (for example, as indicated by a corresponding signal, like of visual and/or acoustic type), the practitioner may retract the catheterization device (so as to extract the needle from the body-part) and may move it away from the patient manually.

With reference instead to block 975 (catheterization device of end-effector type), the control manager stops the actuator as soon as the target region has been reached. The control manager at block 976 commands the corresponding actuator to rotate the support element (with the needle) as above (by the corresponding robotic arm or by the same structure, i.e., curved guide and actuator, described-above for the catheterization device of hand-held type). The control manager at block 978 commands the corresponding actuator to advance the release element (so as to release the needle from the support element) and at the same time commands the corresponding actuator to retract the support element at opposite speed (so as to maintain the needle stationary). The process branches at block 981 according to a type of the release element. If the release element requires a manual intervention to disengage the connector of the needle from it (for example, manually opening the jaws of the clamp), the driving manager at block 984 prompts the practitioner accordingly (for example, via a corresponding visual and/or acoustic signal) and then waits for a confirmation by the practitioner that the operation has been performed (as indicated by a corresponding command entered on the driving device). In response thereto, the process descends into block 987; the same point is also reached directly from block 981 if the connector of the needle automatically disengages from the release element. The driving manager now commands both robotic arms to leave the patient.

Referring back to block 957, if the elapsed time has reached the threshold value (without detecting the target region), the process descends into block 990. At this point, the control manager commands the corresponding actuator to stop advancing the support element; this avoids possible damages to the patient (for example, when the catheterization device has not been positioned correctly, the target region is too deep within the body-part, the patient has moved during the insertion of the needle and so on). The needle and the possible catheter are now extracted from the body-part, retracting the support element (by the corresponding actuator) or the whole catheterization device, and the catheterization device is removed from the patient (with the catheterization device being moved either manually by the practitioner in response to a corresponding visual and/or acoustic signal or automatically by the corresponding robotic arm).

The process then ends at the concentric black/white circles 993 from block 972, from block 987 or from block 990.

### Modifications

In order to satisfy local and specific requirements, a person skilled in the art may apply many logical and/or physical modifications and alterations to the present disclosure. More specifically, although this disclosure has been described with a certain degree of particularity with reference to one or more embodiments thereof, it should be understood that various omissions, substitutions and changes in the form and details as well as other embodiments are possible. Particularly, different embodiments of the present disclosure may be practiced even without the specific details (such as the numerical values) set forth in the preceding description to provide a more thorough understanding thereof; conversely, well-known features may have been omitted or simplified in order not to obscure the description with unnecessary particulars. Moreover, it is expressly intended that specific elements and/or method steps described in connection with any embodiment of the present disclosure may be incorporated in any other embodiment as a matter of general design choice. Moreover, items presented in a same group and different embodiments, examples or alternatives are not to be construed as de facto equivalent to each other (but they are separate and autonomous entities). In any case, each numerical value should be read as modified according to applicable tolerances; particularly, unless otherwise indicated, the terms “substantially”, “about”, “approximately” and the like should be understood as within 10%, preferably 6% and still more preferably 1%. Moreover, each range of numerical values should be intended as expressly specifying any possible number along the continuum within the range (comprising its end points). Ordinal or other qualifiers are merely used as labels to distinguish elements with the same name but do not by themselves connote any priority, precedence or order. The terms include, comprise, have, contain, involve and the like should be intended with an open, non-exhaustive meaning (i.e., not limited to the recited items); the terms based on, dependent on, according to, function of and the like should be intended as a non-exclusive relationship (i.e., with possible further variables involved); the term a/an should be intended as one or more items (unless expressly indicated otherwise); and the term means for (or any means-plus-function formulation) should be intended as any structure adapted or configured for carrying out the relevant function.

For example, an embodiment provides a catheterization device. However, the catheterization device may be of any type (for example, hand-held, end-effector, semi-automatic, completely automatic and so on).

In an embodiment, the catheterization device is for accessing a target region within a body-part of a patient. However, the target region may be of any type within any body-part (for example, a vessel, such as a vein or an artery, a cavity, such as the spinal cavity, a duct, such as the lacrimal duct, an organ, such as the brain or the liver, a tissue, such as adipose tissue, and so on) of any patient (for example, human beings of any age and physiology, animals and so on). Moreover, the target region may be accessed for any purpose (for example, in a peripheral, deep, central and the like catheterization procedure for administering substances, sampling substances, draining substances, inserting medical instruments for intervention or diagnosis, and so on).

In an embodiment, the target region has at least one physical characteristic distinguishing the target region from a rest of the body-part. However, the physical characteristics may be in any number and of any type (for example, electric impedance, pressure, color, temperature, sound and so on).

In an embodiment, the catheterization device comprises a bearing structure. However, the bearing structure may be of any type, shape and size (for example, a chassis, a base, a frame, a casing and so on).

In an embodiment, the catheterization device comprises a slide. However, the slide may be of any type, shape and size (for example, a carriage, a runner, a prism and so no).

In an embodiment, the catheterization device comprises a linear guide arranged on the bearing structure for guiding the slide. However, the linear guide may be of any type (for example, a track, a rail, a channel and the like, with any length and so on) and arranged on the bearing structure in any way (for example, at any position, along any direction and so on).

In an embodiment, the catheterization device comprises a support element for supporting a functional element in a releasable manner. However, the support element may be of any type, size and shape (for example, a carriage, a base, a platform and so on) and it may support the functional element in any releasable manner (for example, the catheter indirectly via the needle, the needle directly and so on); moreover, the functional element may be either the catheter or the needle in every type of catheterization device (for example, hand-held, end-effector and so on).

In an embodiment, the functional element is a catheter. However, the catheter may be of any type, shape and size (for example, PIVC, CVC, PICC, midline and so on).

In an embodiment, in this case the catheter is mounted on a needle. However, the needle may be of any type, shape and size (for example, bipolar, monopolar and so on) and the catheter may be mounted thereon in any releasable manner (for example, press-fitted, snap-fitted, with the needle projecting from the catheter by any distance and so on).

In an embodiment, in this case the needle is mounted on the support element. However, the needle may be mounted on the support element in any manner (for example, press-fitted, snap-fitted, screwed and so on).

In an embodiment, the functional element is the needle being mounted on the support element. However, in this case the needle may be mounted on the support element in any releasable manner (for example, press-fitted, snap-fitted and so on).

In an embodiment, the catheterization device comprises a curved guide arranged on the slide for guiding the support element. However, the curved guide may be of any type (for example, formed by one or more concentric elements, with any shape, such as circular, oval and the like, any curvature radius, length and so on) and arranged on the slide in any way (for example, at any position, along any direction and so on).

In an embodiment, the catheterization device comprises a release element for releasing the functional element from the support element. However, the release element may be of any type, shape and size (for example, a finger, a fork, a clamp a piston and so on) for releasing the functional element in any way (for example, completely or requiring a next manual intervention to disengage the functional element from the release element, pushing or pulling it, acting thereon at any number and type of positions, and so on).

In an embodiment, the catheterization device comprises a first actuator for moving the slide along the linear guide. However, the first actuator may be of any type (for example, an electric motor, a pneumatic cylinder, an electromagnetic device, arranged on the bearing structure or on the slide, and so on) for moving the slide in any way (for example, pushing or pulling it, by any extent and so on).

In an embodiment, the catheterization device comprises a second actuator for moving the release element with respect to the support element. However, the second actuator may be of any type (for example, an electric motor, a pneumatic cylinder, an electromagnetic device and so on) and arranged at any position (for example, on the support element, on the bearing structure and so on) for moving the release element in any way (for example, along any direction, pushing or pulling it, by any extent and so on).

In an embodiment, the catheterization device comprises a third actuator for moving the support element along the curved guide. However, the third actuator may be of any type (for example, any mechanism based on an electric motor, a pneumatic cylinder, an electromagnetic device and so on) for moving the support element in any way (for example, pushing or pulling it, by any extent and so on).

In an embodiment, the catheterization device comprises a sensor for measuring a characteristic signal indicative of a value of the physical characteristic of a material contacting a tip of the needle. However, the sensor may be of any type for measuring any number and type of physical characteristics (for example, electrical, pneumatic, optical, thermal, acoustic and so on).

In an embodiment, the catheterization device comprises a control unit. However, the control unit may be of any type (for example, local thereto, such as a microprocessor, a microcontroller and the like, remote therefrom, such as a separate personal computer, server and the like, and so on).

In an embodiment, the control unit is configured for controlling the first actuator for moving the slide along the linear guide, thereby translating the needle to penetrate the body-part. However, the slide may be moved in any way (for example, with any speed, with or without a maximum travel of any value, in response to any start command, such as entered manually on the catheterization device or on any separate control device via any command, such as a push-button, a lever, a soft-button and the like, generated automatically in response to the detection of the target region, with or without manual confirmation thereof, and so on).

In an embodiment, the control unit is configured for detecting a reaching of the target region by the tip of the needle according to the characteristic signal received from the sensor. However, the reaching of the target region may be detected in any way (for example, as soon as the characteristic signal is indicative of its material, after this condition persists for a pre-defined time, when this is confirmed for two or more physical characteristics, and so on).

In an embodiment, the control unit is configured for controlling the first actuator for stopping said moving the slide in response to the reaching of the target region. However, the first actuator may be stopped in any way (for example, with any delay from the detection of the reaching of the target region, immediately after detecting the reaching of the target region, then controlled to maintain the support element stationary or to move the support element opposite the movement of the functional element, and so on).

In an embodiment, the control unit is configured for controlling the third actuator for moving the support element along the curved guide, thereby rotating the support element to reduce an angle between the needle and the body-part, in response to the reaching of the target region. However, the support element may be moved in any way (for example, with any speed, to reduce the angle by any extent, either in absolute term or in relative term, down to become null, with any advance, at the same time or with any delay with respect to the stopping of the first actuator, and so on).

In an embodiment, the control unit is configured for controlling the second actuator for moving the release element, thereby causing the functional element to release from the support element, in response to a completion of said rotating the support element. However, the release element may be moved in any way (for example, along any linear/non-linear direction, either the same or different with respect to the one of the linear guide, with any speed, with any advance, at the same time or with any delay with respect to the completion of rotating the support element, and so on).

Further embodiments provide additional advantageous features, which may however be omitted at all in a basic implementation. In this respect, it is expressly intended that the features of each of the following embodiments may be combined with the above features either alone or in combination with the features of any number of the other following embodiments.

In an embodiment, the third actuator is arranged on the bearing structure. However, the third actuator may be arranged on the bearing structure in any way (for example, at any position, coupled with the support element in any way, and so on).

In an embodiment, the third actuator comprises a cylinder having a barrel integral with the bearing structure and a piston slidable along the barrel. However, the cylinder may be of any type (for example, pneumatic, mechanic and the like, with any orientation, stroke and so on).

In an embodiment, the third actuator comprises a lever having a fulcrum on the bearing structure dividing the lever into an effort arm with fixed length and a load arm with variable length. However, the lever may be of any type (for example, with the effort arm having any length, the load arm having its length variable within any range and so on) and the variable length of the load arm may be obtained in any way (for example, with a telescopic structure, an elastic structure and so on).

In an embodiment, the piston is hinged to the effort arm and the load arm is hinged to the support element. However, these pairs of elements may be hinged in any way (for example, at any distance from their free ends, down to none, and so on).

In an embodiment, the third actuator is arranged on the slide. However, the third actuator may be arranged on the slide in any way (for example, at any position, coupled with the support element in any way, and so on).

In an embodiment, the third actuator comprises a plurality of pulleys. However, the pulleys may in any number, at any positions and of any type (for example, wheels, shafts/axles and so on).

In an embodiment, the pulleys comprise a driving pulley. However, the driving pulley may be of any type (for example, with any diameter, of smoothed, grooved or geared type, and so on).

In an embodiment, the pulleys comprise one or more idler pulleys. However, the idler pulleys may be in any number and of any type (for example, either the same or different among them and with respect to the driving pulley).

In an embodiment, the third actuator comprises a looped transmission element fitted on the pulleys for being rotated by the driving pulley. However, the looped transmission element may be of any type (for example, a belt of any material, with any section and the like, a chain and so on) and fitted on the pulleys in any way (for example, with any tension, straight or twisted, and so on).

In an embodiment, the third actuator comprises a coupler for coupling the support element with the looped transmission element. However, the coupler may be of any type (for example, a screw, a clip, a clasp and so on).

In an embodiment, the curved guide has a curvature radius for rotating the needle around an axis of rotation passing through the tip of the needle when the support element moves along the curved guide. However, this result may be achieved in any way (for example, with the axis of rotation at any distance from the curved guide, either fixed or adjustable, and so on).

In an embodiment, the support element is adjustable according to a length of the needle for having a distance of the tip of the needle from the curved guide matching the curvature radius of the curved guide. However, the support element may be adjusted in any way (for example, to cover any range of lengths of the needle, by means of a telescopic structure, exchangeable adapters and so on).

In an embodiment, the first actuator is arranged on the bearing structure. However, the first actuator may be arranged on the bearing structure in any way (for example, at any position, coupled with the slide in any way, and so on); however, the possibility of having the first actuator on the slide is not excluded.

In an embodiment, the second actuator is arranged on the slide. However, the second actuator may be arranged on the slide in any way (for example, at any position, coupled with the release element in any way, and so on); however, the possibility of having the second actuator on the bearing structure is not excluded.

In an embodiment, the control unit is configured for controlling the first actuator to maintain the slide stationary during said moving the release element. However, this feature may be used in every type of the catheterization device (for example, hand-held, end-effector, functional element being either the catheter or the needle, and so on), with only this option available or with the possibility of selecting it or the next one statically/dynamically.

In an embodiment, the control unit is configured for controlling the second actuator for moving the release element along a release direction at a release speed and for controlling the first actuator for moving the slide along a retraction direction (opposite the release direction) at a retraction speed (opposite the release speed) during said moving the release element. However, this feature may be used in every type of the catheterization device (for example, hand-held, end-effector, functional element being either the catheter or the needle, and so on), with only this option available or with the possibility of selecting it or the previous one statically/dynamically.

In an embodiment, the catheterization device has a guidance module. However, the guidance module may be of any type (for example, integral or removable, fixed or adjustable, stand-alone or coupled with the control unit, and so on). Moreover, this feature may be present in every type of the catheterization device (for example, handheld, end-effector, functional element being either the catheter or the needle, and so on).

In an embodiment, the guidance module comprises an acquisition unit for acquiring images of an interior of the body-part. However, the acquisition unit may be of any type (for example, for acquiring NIR images, IR images, ultrasound images, with or without a corresponding illumination unit, and so on).

In an embodiment, the guidance module comprises a display unit for displaying a representation of the body-part based on the images. However, the display unit may be of any type (for example, a monitor, a projector and so on) for displaying any representation of the body-part based on the images (for example, the images with the target region enhanced in any way, the images as acquired, the representation of the target region extracted from the images being superimposed on images of an exterior of the body-part or projected onto it, and so on).

In an embodiment, the acquisition unit comprises a camera and a light source of NIR type. However, the camera and the light source may be of any type (for example, a camera based on CCD, ICCD, EMCCD, CMOS, InGaAs or PMT sensors, a light source based on laser, LEDs or UV lamps, and so on).

In an embodiment, the catheterization device comprises means for enhancing a representation of the target region in the images. However, these means may be implemented in any way (for example, in hardware or software, by the control unit of the catheterization device or by any dedicated processing unit of the guidance module, applying algorithmic techniques, deep learning techniques and so on).

In an embodiment, the catheterization device comprises means for mounting the guidance module on the bearing structure in a removable manner. However, these means may be implemented in any way (for example, by a connector, a socket, a housing and so on).

In an embodiment, the catheterization device is of hand-held type with the bearing structure having a foot for resting on the body-part. However, the foot may be of any type (for example, a bottom of a chassis, a dedicated element and so on) for positioning the needle in any way (for example, at any distance from the body-part, down to none, forming any angle with the body-part and so on).

An embodiment provides a robotic system comprising the catheterization device of above. However, the robotic system may be of any type (for example, a manipulator that handles the catheterization device and the possible probe without any physical contact by the practitioner or a co-manipulator (cobot) that collaborates with the practitioner to handle them jointly, automatic or semi-automatic, with local control, remote control and so on).

In an embodiment, the robotic system comprises a robotic arm carrying the catheterization device. However, the robotic arm may be of any type (for example, autonomous or semi-autonomous, of linear type, rotational type, with any number of degrees of freedom and so on).

In an embodiment, the robotic system comprises a driving device for driving the robotic arm to position the catheterization device at the body-part. However, the driving device may be of any type (for example, a physical machine, a virtual machine, a cloud service and so on) for positioning the robotic arm in any way (for example, directly to the position for inserting the needle into the body-part, simply close to the body-part thereby relieving the practitioner from bearing the catheterization device while also allowing him/her to grasp and guide the catheterization device during the catheterization procedure, and so on).

In an embodiment, the robotic system comprises a further robotic arm carrying a probe. However, the further robotic arm may be of any type (for example, either the same or different with respect to the robotic arm) carrying any type of probe (for example, of ultrasound type, infrared type and so on).

In an embodiment, the driving device is configured for determining a position of the target region according to information received from the probe. However, the position of the target region may be determined in any way (for example, in hardware or software, applying algorithmic techniques, deep learning techniques and so on).

In an embodiment, the driving device is configured for driving the robotic arm according to the position of the target region. However, the robotic arm may be driven according to the position of the target region in any way (for example, bringing the tip of the needle to any distance from the body-part, down to none, with the needle forming any angle with the body-part and so on).

Generally, similar considerations apply if the catheterization device and the robotic system each has a different structure, comprises equivalent components (for example, of different materials) or it has other operative characteristics, provided that it remains within the scope of the claims. In any case, every component thereof may be separated into more elements, or two or more components may be combined together into a single element; moreover, each component may be replicated to support the execution of the corresponding operations in parallel. Moreover, unless specified otherwise, any interaction between different components generally does not need to be continuous, and it may be either direct or indirect through one or more intermediaries.

An embodiment provides a control method for controlling the catheterization device of above. In an embodiment, the control method comprises the following steps under the control of a control unit of the catheterization device. In an embodiment, the control method comprises controlling (by the control unit) the first actuator for moving the slide along the linear guide, thereby translating the needle to penetrate the body-part. In an embodiment, the control method comprises detecting (by the control unit) a reaching of the target region by the tip of the needle according to the characteristic signal received from the sensor. In an embodiment, the control method comprises controlling (by the control unit) the first actuator for stopping said moving the slide in response to the reaching of the target region. In an embodiment, the control method comprises controlling (by the control unit) the third actuator for moving the support element along the curved guide, thereby rotating the support element to reduce an angle between the needle and the body-part, in response to the reaching of the target region. In an embodiment, the control method comprises controlling (by the control unit) the second actuator for moving the release element, thereby causing the functional element to release from the support element, in response to a completion of said rotating the support element. However, the same considerations pointed out above with respect to the features of the catheterization device apply to the corresponding steps of the method as well.

Generally, similar considerations apply if the same solution is implemented with an equivalent method (by using similar steps with the same functions of more steps or portions thereof, removing some non-essential steps or adding further optional steps), provided that it remains within the scope of the claims; moreover, the steps may be performed in a different order, concurrently or in an interleaved way (at least in part).

An embodiment provides a computer program configured for causing the catheterization device to perform the method of above when the computer program is executed on the control unit of the catheterization device. An embodiment provides a computer program product comprising one or more computer readable storage media having program instructions collectively stored on the computer readable storage media, the program instructions readable by the control unit of the catheterization device to cause the catheterization device to perform the same method. However, the software program may be implemented as a stand-alone module, as a plug-in for a pre-existing software program (for example, a control manager of the catheterization device) or even directly in the latter, and it may be used on any control unit (see above).

Generally, the (software) program may be structured in a different way, or additional modules or functions may be provided. The program may take any form suitable to be used by the control unit, thereby configuring it to perform the desired operations; particularly, the program may be in the form of external or resident software, firmware, or microcode (either in object code or in source code), for example, to be compiled or interpreted. Moreover, it is possible to provide the program on any computer readable storage medium. The storage medium is any tangible medium (different from transitory signals per se) that may retain and store instructions for use by the control unit. For example, the storage medium may be of the electronic, magnetic, optical, electromagnetic, infrared, or semiconductor type; examples of such storage medium are embedded memories (where the program may be pre-loaded), removable memory cards, memory keys (for example, USB), and the like. The program may be downloaded to the control unit from the storage medium or via a network (for example, the Internet, a wide area network and/or a local area network comprising transmission cables, optical fibers, wireless connections, network devices). In any case, the solution according to an embodiment of the present disclosure lends itself to be implemented even with a hardware structure (for example, by electronic circuits integrated on one or more chips of semiconductor material), or with a combination of software and hardware suitably programmed or otherwise configured.

An embodiment provides a catheterization method for inserting a catheter into a target region within a body-part of a patient. However, the catheterization method may be used for performing any medical procedure on any target region within any body-part of any patient (see above).

In an embodiment, the catheterization method comprises placing the catheterization device of above at the target region. However, the catheterization device may be placed at the target region in anyway (for example, manually, automatically and so on).

In an embodiment, the catheterization method comprises controlling the catheterization device according to the above method. However, the catheterization device may be controlled in any way (for example, locally, remotely and so on).

1. A catheterization device for accessing a target region within a body-part of a patient, the target region having at least one physical characteristic distinguishing the target region from a rest of the body-part, wherein the catheterization device comprises:
a bearing structure,
a slide
a linear guide arranged on the bearing structure for guiding the slide,
a support element for supporting a functional element in a releasable manner, wherein the functional element is a catheter being mounted on a needle with the needle being mounted on the support element or wherein the functional element is the needle being mounted on the support element,
a curved guide arranged on the slide for guiding the support element,
a release element for releasing the functional element from the support element,
a first actuator for moving the slide along the linear guide
a second actuator for moving the release element with respect to the support element,
a third actuator for moving the support element along the curved guide,
a sensor for measuring a characteristic signal indicative of a value of the physical characteristic of a material contacting a tip of the needle, and
a control unit configured for:
i) controlling the first actuator for moving the slide along the linear guide, thereby translating the needle to penetrate the body-part,
ii) detecting a reaching of the target region by the tip of the needle according to the characteristic signal received from the sensor,
iii) controlling the first actuator for stopping said moving the slide and the third actuator for moving the support element along the curved guide, thereby rotating the support element to reduce an angle between the needle and the body-part, in response to the reaching of the target region, and
iv) controlling the second actuator for moving the release element thereby causing the functional element to release from the support element, in response to a completion of said rotating the support element.

2. The catheterization device according to claim 1, wherein the third actuator is arranged on the bearing structure

3. The catheterization device according to claim 2, wherein the third actuator comprises a cylinder, having a barrel integral with the bearing structure and a piston slidable along the barrel, and a lever, having a fulcrum on the bearing structure dividing the lever into an effort arm with fixed length and a load arm with variable length, the piston being hinged to the effort arm and the load arm being hinged to the support element.

4. The catheterization device according to claim 1, wherein the third actuator is arranged on the slide.

5. The catheterization device according to claim 4, wherein the third actuator comprises a plurality of pulleys, comprising a driving pulley and one or more idler pulleys, a looped transmission element fitted on the pulleys for being rotated by the driving pulley, and a coupler for coupling the support element with the looped transmission element.

6. The catheterization device according to claim 1, wherein the curved guide has a curvature radius for rotating the needle around an axis of rotation passing through the tip of the needle when the support element is moved along the curved guide.

7. The catheterization device according to claim 6, wherein the support element is adjustable according to a length of the needle for having a distance of the tip of the needle from the curved guide matching the curvature radius of the curved guide.

8. The catheterization device according to claim 1, wherein the first actuator is arranged on the bearing structure and wherein the second actuator is arranged on the slide.

9. The catheterization device according to claim 1, wherein the control unit is configured for controlling the first actuator to maintain the slide stationary during said moving the release element.

10. The catheterization device according to claim 1, wherein the control unit is configured for controlling the second actuator for moving the release element along a release direction at a release speed and for controlling the first actuator for moving the slide along a retraction direction opposite the release direction at a retraction speed opposite the release speed during said moving the release element.

11. The catheterization device according to claim 1, wherein the catheterization device has a guidance module comprising an acquisition unit for acquiring images of an interior of the body-part and a display unit for displaying a representation of the body-part based on the images.

12. The catheterization device according to claim 11, wherein the acquisition unit comprises a camera and a light source of NIR type, and wherein the control unit is configured for enhancing a representation of the target region in the images.

13. The catheterization device according to claim 11, wherein the catheterization device comprises a mounting structure for mounting the guidance module on the bearing structure in a removable manner.

14. The catheterization device according to claim 1, wherein the catheterization device is of hand-held type with the bearing structure having a foot for resting on the body-part.

15. A robotic system comprising the catheterization device according to claim 1, a robotic arm carrying the catheterization device and a driving device for driving the robotic arm to position the catheterization device at the body-part.

16. The robotic system according to claim 15, wherein the robotic system comprises a further robotic arm carrying a probe, the driving device being configured for determining a position of the target region according to information received from the probe and for driving the robotic arm according to the position of the target region.

17. A control method for controlling a catheterization device for accessing a target region within a body-part of a patient, the target region having at least one physical characteristic distinguishing the target region from a rest of the body-part, wherein the catheterization device comprises a bearing structure, a slide, a linear guide arranged on the bearing structure for guiding the slide, a support element for supporting a functional element in a releasable manner, wherein the functional element is a catheter, being mounted on a needle with the needle being mounted on the support element or wherein the functional element is the needle being mounted on the support element, a curved guide arranged on the slide for guiding the support element, a release element for releasing the functional element from the support element, a first actuator for moving the slide along the linear guide, a second actuator for moving the release element with respect to the support element, a third actuator for moving the support element along the curved guide, a sensor for measuring a characteristic signal indicative of a value of the physical character of a material contacting a tip of the needle, and a control unit, wherein the control method comprises, under the control of the control unit:
controlling, by the control unit, the first actuator for moving the slide along the linear guide, thereby translating the needle to penetrate the body-part,
detecting, by the control unit, a reaching of the target region by the tip of the needle according to the characteristic signal received from the sensor,
controlling, by the control unit, the first actuator for stopping said moving the slide and the third actuator for moving the support element along the curved guide, thereby rotating the support element to reduce an angle between the needle and the body-part, in response to the reaching of the target region, and
controlling, by the control unit, the second actuator for moving the release element, thereby causing the functional element to release from the support element, in response to a completion of said rotating the support element.

18. (canceled)

19. A computer program product comprising one or more computer readable storage media having program instructions collectively stored on the computer readable storage media, the program instructions readable by a control unit of a catheterization device to cause the catheterization device to perform a method for accessing a target region within a body-part of a patient, the target region having at least one physical characteristic distinguishing the target region from a rest of the body-part, wherein the catheterization device comprises a bearing structure, a slide, a linear guide arranged on the beating structure for guiding the slide, a support for supporting a functional element in a releasable manner, wherein the functional element is a catheter being mounted on a needle with the needle being mounted on the support element or wherein the functional element is the needle being mounted on the support element, a curved guide arranged on the slide for guiding the support element, a release element for releasing the functional element from the support element, a first actuator for moving the slide along the linear guide, a second actuator for moving the release element with respect to the support element, a third actuator for moving the support element along the curved guide, a sensor measuring a characteristic signal indicative of a value of the physical characteristic of a material contacting a tip of the needle, and a control unit, wherein the method comprises:
controlling the first actuator for moving the slide along the linear guide, thereby translating the needle to penetrate the body-part,
detecting a reaching of the target region by the tip of the needle according to the characteristic signal received from the sensor,
controlling, the first actuator for stopping said moving the slide and the third actuator for moving the support element along the curved guide, thereby rotating the support element to reduce an angle between the needle and the body-part, in response to the reaching of the target region, and
controlling, the second actuator for moving the release element, thereby causing the functional element to release from the support element, in response to completion of said rotating the support element.

20. A catheterization method for inserting a catheter into a target region within a body-part of a patient, wherein the catheterization method comprises:
placing the catheterization device of claim 1 at the target region,
controlling, by the unit, the first actuator for moving the slide along the linear guide, thereby translating the needle to penetrate the body-part,
detecting, by the control unit, a reaching of the target region by the tip of the needle according to he characteristic signal received from the sensor,
controlling by the control unit, the first actuator for stopping said moving the slide and the third actuator moving the support element along the curved guide, thereby rotating the support element to reduce an angle between the needle and the body-part, in response to the reaching of the target region, and
controlling, by the control unit, the second actuator for moving the release element, thereby causing the functional element to release from the support element, in response to a completion of said rotating the support element.
